(19) United States
(12) Patent Application Publication
(10) Pub. No.: US 2007/0092875 A1
(43) Pub. Date: Apr. 26, 2007

(54) Map2k6 as modifier of branching morphogenesis and methods of use

(75) Inventor: Gregory D Plowman, San Carlos, CA (US)

Correspondence Address: MCDONNELL BOEHNEN HULBERT @ BERGHOFF LLP, 300 SOUTH WACKER DRIVE, SUITE 3100, CHICAGO, IL, 60606, US

(73) Assignee: EXELIXIS, INC., South San Francisco, CA (US)

(21) Appl. No.: 10/532,432

(22) PCT Filed: Oct. 22, 2003
(86) PCT No.: PCT/US03/33489

§ 371 Date: Oct. 3, 2006

Related U.S. Application Data
(60) Provisional application No. 60/420,554, filed on Oct. 23, 2002.

(51) Int. Cl. C12Q 1/68; G01N 33/574

(52) U.S. Cl. 435/6; 435/7.23

(57) ABSTRACT

Human MAP2K6 genes are identified as modulators of branching morphogenesis, and thus are therapeutic targets for disorders associated with defective branching morphogenesis function. Methods for identifying modulators of branching morphogenesis, comprising screening for agents that modulate the activity of MAP2K6 are provided.

### REFERENCE TO RELATED APPLICATIONS

This application claims priority to U.S. provisional patent application 60/420,554 filed Oct. 23, 2002. The contents of the prior application are hereby incorporated in their entirety.

### BACKGROUND OF THE INVENTION

Several essential organs (e.g., lungs, kidney, lymphatic system and vasculature) are made up of complex networks of tube-like structures that serve to transport and exchange fluids, gases, nutrients and waste. The formation of these complex branched networks occurs by the evolutionarily conserved process of branching morphogenesis, in which successive ramification occurs by sprouting, pruning and remodeling of the network. During human embryogenesis, blood vessels develop via two processes: vasculogenesis, whereby endothelial cells are born from progenitor cell types; and angiogenesis, in which new capillaries sprout from existing vessels.

Branching morphogenesis encompasses many cellular processes, including proliferation, survival/apoptosis, migration, invasion, adhesion, aggregation and matrix remodeling. Numerous cell types contribute to branching morphogenesis, including endothelial, epithelial and smooth muscle cells, and monocytes. Gene pathways that modulate the branching process function both within the branching tissues as well as in other cells, e.g., certain monocytes can promote an angiogenic response even though they may not directly participate in the formation of the branch structures.

An increased level of angiogenesis is central to several human disease pathologies, including rheumatoid arthritis and diabetic retinopathy, and, significantly, to the growth, maintenance and metastasis of solid tumors (for detailed reviews see Liotta L A et al, 1991 Cell 64:327-336; Folkman J., 1995 Nature Medicine 1:27-31; Hanahan D and Folkman J, 1996 Cell 86:353-364). Impaired angiogenesis figures prominently in other human diseases, including heart disease, stroke, infertility, ulcers and scleroderma.

The transition from dormant to active blood vessel formation involves modulating the balance between angiogenic stimulators and inhibitors. Under certain pathological circumstances an imbalance arises between local inhibitory controls and angiogenic inducers resulting in excessive angiogenesis, while under other pathological conditions an imbalance leads to insufficient angiogenesis. This delicate equilibrium of pro- and anti-angiogenic factors is regulated by a complex interaction between the extracellular matrix, endothelial cells, smooth muscle cells, and various other cell types, as well as environmental factors such as oxygen demand within tissues. The lack of oxygen (hypoxia) in and around wounds and solid tumors is thought to provide a key driving force for angiogenesis by regulating a number of angiogenic factors, including Hypoxia Induced Factor alpha (HIF1 alpha) (Richard D E et al., Biochem Biophys Res Commun. 1999 Dec. 29; 266(3):718-22). HIF1 in turn regulates expression of a number of growth factors including Vascular Endothelial Growth Factor (VEGF) (Connolly D T, J Cell Biochem 1991 November; 47(3):219-23). Various VEGF ligands and receptors are vital regulators of endothelial cell proliferation, survival, vessel permeability and sprouting, and lymphangiogenesis (Neufeld G et al., FASEB J 1999 January; 13(1):9-22; Stacker S A et al., Nature Medicine 2001 7:186-191; Skobe M, et al., Nature Medicine 2001 7:192-198; Makinen T, et al., Nature Medicine 2001 7:199-205).

Most known angiogenesis genes, their biochemical activities, and their organization into signaling pathways are employed in a similar fashion during angiogenesis in human, mouse and Zebrafish, as well as during branching morphogenesis of the Drosophila trachea. Accordingly, Drosophila tracheal development and zebrafish vascular development provide useful models for studying mammalian angiogenesis (Sutherland D et al., Cell 1996, 87:1091-101; Roush W, Science 1996, 274:2011; Skaer H., Curr Biol 1997, 7:R238-41; Metzger R J, Krasnow M A. Science. 1999. 284:1635-9; Roman B L, and Weinstein B M. Bioessays 2000, 22:882-93).

Mitogen-activated protein kinases (MAPKs) are key components in various cellular signal transduction pathways that affect growth factor-induced proliferation, gene expression, and compensation for environmental changes (Seger, R.; and Krebs, E. G. (1995) FASEB J. 9: 726-735). To become activated, MAPKs require dual phosphorylation on threonine and tyrosine residues, an activation step carried out by MAPK kinases (MAP2Ks, also called MKKs or MEKs). MAP2K6 (Mitogen-activated protein kinase 6) is a member of the MKK pathway, and is involved in intracellular signalling pathways leading toward activation of the p38 MAP kinase (Han, J. et al (1996) J. Biol. Chem. 271: 2886-2891). During mammalian development, electrical activity promotes the calcium-dependent survival of neurons that have made appropriate synaptic connections. Calcium influx into cerebellar neurons triggers the activation of the MAP2K6-p38 MAP kinase cascade, where the p38 MAP kinase then phosphorylates and activates MEF2s. Once activated by this calcium-dependent p38 MAP kinase signaling pathway, MEF2 can regulate the expression of genes that are critical for survival of newly differentiated neurons (Mao, Z. et al (1999) Science 286: 785-790). MAP2K6 is involved in promoting cell cycle arrest and protection from apoptosis in response to a variety of insults (Wang, X., et al (2000) Mol Cell Biol 20:4543-52). The ability to manipulate and screen the genomes of model organisms such as Drosophila and zebrafish provides a powerful means to analyze biochemical processes that, due to significant evolutionary conservation of genes, pathways, and cellular processes, have direct relevance to more complex vertebrate organisms.

Short life cycles and powerful forward and reverse genetic tools available for both Zebrafish and Drosophila allow rapid identification of critical components of pathways controlling branching morphogenesis. Given the evolutionary conservation of gene sequences and molecular pathways, the human orthologs of model organism genes can be utilized to modulate branching morphogenesis pathways, including angiogenesis.

All references cited herein, including patents, patent applications, publications, and sequence information in referenced Genbank identifier numbers, are incorporated herein in their entireties.

### SUMMARY OF THE INVENTION

We have discovered genes that modify branching morphogenesis in zebrafish (Danio rerio), and identified their human orthologs. One identified zebrafish modifier was Dr_map2k3, and its identified human ortholog is hereinafter referred to as Mitogen-activated protein kinase 6 (MAP2K6). The invention provides methods for utilizing these branching morphogenesis modifier genes and polypeptides to identify MAP2K6-modulating agents that are candidate therapeutic agents that can be used in the treatment of disorders associated with defective or impaired branching morphogenesis function and/or MAP2K6 function. Preferred MAP2K6-modulating agents specifically bind to MAP2K6 polypeptides and restore branching morphogenesis function. Other preferred MAP2K6-modulating agents are nucleic acid modulators such as antisense oligomers and RNAi that repress MAP2K6 gene expression or product activity by, for example, binding to and inhibiting the respective nucleic acid (i.e. DNA or mRNA).

MAP2K6 modulating agents may be evaluated by any convenient in vitro or in vivo assay for molecular interaction with a MAP2K6 polypeptide or nucleic acid. In one embodiment, candidate MAP2K6 modulating agents are tested with an assay system comprising a MAP2K6 polypeptide or nucleic acid. Agents that produce a change in the activity of the assay system relative to controls are identified as candidate branching morphogenesis modulating agents. The assay system may be cell-based or cell-free. MAP2K6-modulating agents include MAP2K6 related proteins (e.g. dominant negative mutants, and biotherapeutics); MAP2K6-specific antibodies; MAP2K6-specific antisense oligomers and other nucleic acid modulators; and chemical agents that specifically bind to or interact with MAP2K6 or compete with MAP2K6 binding partner (e.g. by binding to a MAP2K6 binding partner). In one specific embodiment, a small molecule modulator is identified using a kinase assay. In specific embodiments, the screening assay system is selected from a binding assay, an apoptosis assay, a cell proliferation assay, an angiogenesis assay, a hypoxic induction assay, a tubulogenesis assay, a cell adhesion assay, and a sprouting assay.

In another embodiment of the invention, the assay system comprises cultured cells or a non-human animal expressing MAP2K6, and the assay system detects an agent-biased change in branching morphogenesis, including angiogenesis. Events detected by cell-based assays include cell proliferation, cell cycling, apoptosis, tubulogenesis, cell migration, and response to hypoxic conditions. For assays that detect tubulogenesis or cell migration, the assay system may comprise the step of testing the cellular response to stimulation with at least two different pro-angiogenic agents. Alternatively, tubulogenesis or cell migration may be detected by stimulating cells with an inflammatory angiogenic agent. In specific embodiments, the animal-based assay is selected from a matrix implant assay, a xenograft assay, a hollow fiber assay, or a transgenic tumor assay.

In another embodiment, candidate branching morphogenesis modulating agents that have been identified in cell-free or cell-based assays are further tested using a second assay system that detects changes in an activity associated with branching morphogenesis. In a specific embodiment, the second assay detects an agent-biased change in an activity associated with angiogenesis. The second assay system may use cultured cells or non-human animals. In specific embodiments, the secondary assay system uses non-human animals, including animals predetermined to have a disease or disorder implicating branching morphogenesis, including increased or impaired angiogenesis or solid tumor metastasis.

The invention further provides methods for modulating the MAP2K6 function and/or branching morphogenesis in a mammalian cell by contacting the mammalian cell with an agent that specifically binds a MAP2K6 polypeptide or nucleic acid. The agent may be a small molecule modulator, a nucleic acid modulator, or an antibody and may be administered to a mammalian animal predetermined to have a pathology associated branching morphogenesis.

### DETAILED DESCRIPTION OF THE INVENTION

Genetic screens were designed to identify modifiers of branching morphogenesis in zebrafish. We used a screen based on antisense technologies to identify genes whose disruption produced vascular defects in zebrafish. Briefly, and as further described in the Examples, one-cell stage embryos were treated with antisense morpholino oligonucleotides (PMOs) that targeted a large number of predicted zebrafish genes. Treated animals were fixed at the larval stage, and alkaline phosphatase staining was used to visualize blood vessel formation. Antisense knock-down of the zebrafish Dr-map2k3 produced specific vascular defects. Thus, Dr-map2k3gene was identified as a modifier of branching morphogenesis. Accordingly, vertebrate orthologs of this modifier, and preferably the human orthologs, MAP2K6 genes (i.e., nucleic acids and polypeptides) are attractive drug targets for the treatment of pathologies associated with a defective branching morphogenesis signaling pathway, such as cancer.

In vitro and in vivo methods of assessing MAP2K6 function are provided herein. Modulation of the MAP2K6 or their respective binding partners is useful for understanding the association of branching morphogenesis and its members in normal and disease conditions and for developing diagnostics and therapeutic modalities for branching morphogenesis related pathologies. MAP2K6-modulating agents that act by inhibiting or enhancing MAP2K6 expression, directly or indirectly, for example, by affecting a MAP2K6 function such as enzymatic (e.g., catalytic) or binding activity, can be identified using methods provided herein. MAP2K6 modulating agents are useful in diagnosis, therapy and pharmaceutical development.

As used herein, branching morphogenesis encompasses the numerous cellular process involved in the formation of branched networks, including proliferation, survival/apoptosis, migration, invasion, adhesion, aggregation and matrix remodeling. As used herein, pathologies associated with branching morphogenesis encompass pathologies where branching morphogenesis contributes to maintaining the healthy state, as well as pathologies whose course may be altered by modulation of the branching morphogenesis.

Nucleic Acids and Polypeptides of the Invention

Sequences related to MAP2K6 nucleic acids and polypeptides that can be used in the invention are disclosed in Genbank (referenced by Genbank identifier (GI) number) as GI#s 14589899 (SEQ ID NO:1) and 14589901 (SEQ ID NO:2) for nucleic acid, and GI# 14589900 (SEQ ID NO:3) for polypeptide sequences.

The term “MAP2K6 polypeptide” refers to a full-length MAP2K6 protein or a functionally active fragment or derivative thereof. A “functionally active” MAP2K6 fragment or derivative exhibits one or more functional activities associated with a full-length, wild-type MAP2K6 protein, such as antigenic or immunogenic activity, enzymatic activity, ability to bind natural cellular substrates, etc. The functional activity of MAP2K6 proteins, derivatives and fragments can be assayed by various methods known to one skilled in the art (Current Protocols in Protein Science (1998) Coligan et al., eds., John Wiley & Sons, Inc., Somerset, N.J.) and as further discussed below. In one embodiment, a functionally active MAP2K6 polypeptide is a MAP2K6 derivative capable of rescuing defective endogenous MAP2K6 activity, such as in cell based or animal assays; the rescuing derivative may be from the same or a different species. For purposes herein, functionally active fragments also include those fragments that comprise one or more structural domains of a MAP2K6, such as a kinase domain or a binding domain. Protein domains can be identified using the PFAM program (Bateman A., et al., Nucleic Acids Res, 1999, 27:260-2). For example, the kinase domain (PFAM 00069) of MAP2K6 from GI# 14589900 (SEQ ID NO:3) is located at approximately amino acid residues 53 to 314. Methods for obtaining MAP2K6 polypeptides are also further described below. In some embodiments, preferred fragments are functionally active, domain-containing fragments comprising at least 25 contiguous amino acids, preferably at least 50, more preferably 75, and most preferably at least 100 contiguous amino acids of SEQ ID NO:3 (MAP2K6). In further preferred embodiments, the fragment comprises the entire kinase (functionally active) domain.

The term “MAP2K6 nucleic acid” refers to a DNA or RNA molecule that encodes a MAP2K6 polypeptide. Preferably, the MAP2K6 polypeptide or nucleic acid or fragment thereof is from a human, but can also be an ortholog, or derivative thereof with at least 70% sequence identity, preferably at least 80%, more preferably 85%, still more preferably 90%, and most preferably at least 95% sequence identity with human MAP2K6. Methods of identifying orthlogs are known in the art. Normally, orthologs in different species retain the same function, due to presence of one or more protein motifs and/or 3-dimensional structures. Orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen M A and Bork P, Proc Natl Acad Sci (1998) 95:5849-5856; Huynen M A et al., Genome Research (2000) 10:1204-1210). Programs for multiple sequence alignment, such as CLUSTAL (Thompson J D et al, 1994, Nucleic Acids Res 22:4673-4680) may be used to highlight conserved regions and/or residues of orthologous proteins and to generate phylogenetic trees. In a phylogenetic tree representing multiple homologous sequences from diverse species (e.g., retrieved through BLAST analysis), orthologous sequences from two species generally appear closest on the tree with respect to all other sequences from these two species. Structural threading or other analysis of protein folding (e.g., using software by ProCeryon, Biosciences, Salzburg, Austria) may also identify potential orthologs. In evolution, when a gene duplication event follows speciation, a single gene in one species, such as zebrafish, may correspond to multiple genes (paralogs) in another, such as human. As used herein, the term “orthologs” encompasses paralogs. As used herein, “percent (%) sequence identity” with respect to a subject sequence, or a specified portion of a subject sequence, is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2.0a19 (Altschul et al., J. Mol. Biol. (1997) 215:403-410) with all the search parameters set to default values. The HSP S and HSP S2 parameters are dynamic values and are established by the program itself depending upon the composition of the particular sequence and composition of the particular database against which the sequence of interest is being searched. A % identity value is determined by the number of matching identical nucleotides or amino acids divided by the sequence length for which the percent identity is being reported. “Percent (%) amino acid sequence similarity” is determined by doing the same calculation as for determining % amino acid sequence identity, but including conservative amino acid substitutions in addition to identical amino acids in the computation.

A conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine, methionine, and valine; interchangeable polar amino acids are glutamine and asparagine; interchangeable basic amino acids are arginine, lysine and histidine; interchangeable acidic amino acids are aspartic acid and glutamic acid; and interchangeable small amino acids are alanine, serine, threonine, cysteine and glycine.

Alternatively, an alignment for nucleic acid sequences is provided by the local homology algorithm of Smith and Waterman (Smith and Waterman, 1981, Advances in Applied Mathematics 2:482-489; database: European Bioinformatics Institute; Smith and Waterman, 1981, J. of Molec. Biol., 147:195-197; Nicholas et al., 1998, “A Tutorial on Searching Sequence Databases and Sequence Scoring Methods” (www.psc.edu) and references cited therein; W. R. Pearson, 1991, Genomics 11:635-650). This algorithm can be applied to amino acid sequences by using the scoring matrix developed by Dayhoff (Dayhoff: Atlas of Protein Sequences and Structure, M. O. Dayhoff ed., 5 suppl. 3:353-358, National Biomedical Research Foundation, Washington, D.C., USA), and normalized by Gribskov (Gribskov 1986 Nucl. Acids Res. 14(6):6745-6763). The Smith-Waterman algorithm may be employed where default parameters are used for scoring (for example, gap open penalty of 12, gap extension penalty of two). From the data generated, the “Match” value reflects “sequence identity.”

Derivative nucleic acid molecules of the subject nucleic acid molecules include sequences that hybridize to the nucleic acid sequence of MAP2K6. The stringency of hybridization can be controlled by temperature, ionic strength, pH, and the presence of denaturing agents such as formamide during hybridization and washing. Conditions routinely used are set out in readily available procedure texts (e.g., Current Protocol in Molecular Biology, Vol. 1, Chap. 2.10, John Wiley & Sons, Publishers (1994); Sambrook et al., Molecular Cloning, Cold Spring Harbor (1989)). In some embodiments, a nucleic acid molecule of the invention is capable of hybridizing to a nucleic acid molecule containing the nucleotide sequence of MAP2K6 under high stringency hybridization conditions that are: prehybridization of filters containing nucleic acid for 8 hours to overnight at 65° C. in a solution comprising 6× single strength citrate (SSC) (1×SSC is 0.15 M NaCl, 0.015 M Na citrate; pH 7.0), 5× Denhardt's solution, 0.05% sodium pyrophosphate and 100 μg/ml herring sperm DNA; hybridization for 18-20 hours at 65° C. [symbol] a solution containing 6×SSC, 1× Denhardt's solution, 100 μg/ml yeast tRNA and [symbol] sodium pyrophosphate; and washing of filters at 65° C. for 1 h in a solution containing 0.1×SSC and 0.1% SDS (sodium dodecyl sulfate).

In other embodiments, moderately stringent hybridization conditions are used that are: pretreatment of filters containing nucleic acid for 6 h at 40° C. in a solution containing 35% formamide, 5×SSC, 50 mM Tris-HCl (pH7.5), 5 mM EDTA, 0.1% PVP, 0.1% Ficoll, 1% BSA, and 500 μg/ml denatured salmon sperm DNA; hybridization for 18-20 h at 40° C. in a solution containing 35% formamide, 5×SSC, 50 mM Tris-HCl (pH7.5), 5 mM EDTA, 0.02% PVP, 0.02% Ficoll, 0.2% BSA, 100 μg/ml salmon sperm DNA, and 10% (wt/vol) dextran sulfate; followed by washing twice for 1 hour at 55° C. in a solution containing 2×SSC and 0.1% SDS.

Alternatively, low stringency conditions can be used that are: incubation for 8 hours to overnight at 37° C. in a solution comprising 20% formamide, 5×SSC, 50 mM sodium phosphate (pH 7.6), 5× Denhardt's solution, 10% dextran sulfate, and 20 μg/ml denatured sheared salmon sperm DNA; hybridization in the same buffer for 18 to 20 hours; and washing of filters in 1×SSC at about 37° C. for 1 hour.

Isolation, Production, Expression, and Mis-Expression of MAP2K6 Nucleic Acids and Polypeptides

MAP2K6 nucleic acids and polypeptides, are useful for identifying and testing agents that modulate MAP2K6 function and for other applications related to the involvement of MAP2K6 in branching morphogenesis. MAP2K6 nucleic acids and derivatives and orthologs thereof may be obtained using any available method. For instance, techniques for isolating cDNA or genomic DNA sequences of interest by screening DNA libraries or by using polymerase chain reaction (PCR) are well known in the art. In general, the particular use for the protein will dictate the particulars of expression, production, and purification methods. For instance, production of proteins for use in screening for modulating agents may require methods that preserve specific biological activities of these proteins, whereas production of proteins for antibody generation may require structural integrity of particular epitopes. Expression of proteins to be purified for screening or antibody production may require the addition of specific tags (e.g., generation of fusion proteins). Overexpression of a MAP2K6 protein for assays used to assess MAP2K6 function, such as involvement in cell cycle regulation or hypoxic response, may require expression in eukaryotic cell lines capable of these cellular activities. Techniques for the expression, production, and purification of proteins are well known in the art; any suitable means therefore may be used (e.g., Higgins S J and Hames B D (eds.) Protein Expression: A Practical Approach, Oxford University Press Inc., New York 1999; Stanbury P F et al., Principles of Fermentation Technology, 2^{nd} edition, Elsevier Science, N.Y., 1995; Doonan S (ed.) Protein Purification Protocols, Humana Press, N.J., 1996; Coligan J E et al, Current Protocols in Protein Science (eds.), 1999, John Wiley & Sons, New York). In particular embodiments, recombinant MAP2K6 is expressed in a cell line known to have defective branching morphogenesis function. The recombinant cells are used in cell-based screening assay systems of the invention, as described further below.

The nucleotide sequence encoding a MAP2K6 polypeptide can be inserted into any appropriate expression vector. The necessary transcriptional and translational signals, including promoter/enhancer element, can derive from the native MAP2K6 gene and/or its flanking regions or can be heterologous. A variety of host-vector expression systems may be utilized, such as mammalian cell systems infected with virus (e.g. vaccinia virus, adenovirus, etc.); insect cell systems infected with virus (e.g. baculovirus); microorganisms such as yeast containing yeast vectors, or bacteria transformed with bacteriophage, plasmid, or cosmid DNA. An isolated host cell strain that modulates the expression of, modifies, and/or specifically processes the gene product may be used.

To detect expression of the MAP2K6 gene product, the expression vector can comprise a promoter operably linked to a MAP2K6 gene nucleic acid, one or more origins of replication, and, one or more selectable markers (e.g. thymidine kinase activity, resistance to antibiotics, etc.). Alternatively, recombinant expression vectors can be identified by assaying for the expression of the MAP2K6 gene product based on the physical or functional properties of the MAP2K6 protein in in vitro assay systems (e.g. immunoassays).

The MAP2K6 protein, fragment, or derivative may be optionally expressed as a fusion, or chimeric protein product (i.e. it is joined via a peptide bond to a heterologous protein sequence of a different protein), for example to facilitate purification or detection. A chimeric product can be made by ligating the appropriate nucleic acid sequences encoding the desired amino acid sequences to each other using standard methods and expressing the chimeric product. A chimeric product may also be made by protein synthetic techniques, e.g. by use of a peptide synthesizer (Hunkapiller et al., Nature (1984) 310:105-111).

Once a recombinant cell that expresses the MAP2K6 gene sequence is identified, the gene product can be isolated and purified using standard methods (e.g. ion exchange, affinity, and gel exclusion chromatography; centrifugation; differential solubility; electrophoresis). Alternatively, native MAP2K6 proteins can be purified from natural sources, by standard methods (e.g. immunoaffinity purification). Once a protein is obtained, it may be quantified and its activity measured by appropriate methods, such as immunoassay, bioassay, or other measurements of physical properties, such as crystallography.

The methods of this invention may also use cells that have been engineered for altered expression (mis-expression) of MAP2K6 or other genes associated with branching morphogenesis. As used herein, mis-expression encompasses ectopic expression, over-expression, under-expression, and non-expression (e.g. by gene knock-out or blocking expression that would otherwise normally occur).

Genetically Modified Animals

Animal models that have been genetically modified to alter MAP2K6 expression may be used in in vivo assays to test for activity of a candidate branching morphogenesis modulating agent, or to further assess the role of MAP2K6 in a branching morphogenesis process such as apoptosis or cell proliferation. Preferably, the altered MAP2K6 expression results in a detectable phenotype, such as decreased or increased levels of cell proliferation, angiogenesis, or apoptosis compared to control animals having normal MAP2K6 expression. The genetically modified animal may additionally have altered branching morphogenesis expression (e.g. branching morphogenesis knockout). Preferred genetically modified animals are mammals such as primates, rodents (preferably mice or rats), among others. Preferred non-mammalian species include zebrafish, C. elegans, and Drosophila. Preferred genetically modified animals are transgenic animals having a heterologous nucleic acid sequence present as an extrachromosomal element in a portion of its cells, i.e. mosaic animals (see, for example, techniques described by Jakobovits, 1994, Curr. Biol. 4:761-763) or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal.

Methods of making transgenic animals are well-known in the art (for transgenic mice see Brinster et al., Proc. Nat. Acad. Sci. USA 82: 4438-4442 (1985), U.S. Pat. Nos. 4,736,866 and 4,870,009, both by Leder et al., U.S. Pat. No. 4,873,191 by Wagner et al., and Hogan, B., Manipulating the Mouse Embryo, Cold Spring Harbor Laboratory Press, Cold Spring Harbor, N.Y., (1986); for particle bombardment see U.S. Pat. No., 4,945,050, by Sandford et al.; for transgenic Drosophila see Rubin and Spradling, Science (1982) 218:348-53 and U.S. Pat. No. 4,670,388; for transgenic insects see Berghammer A. J. et al., A Universal Marker for Transgenic Insects (1999) Nature 402:370-371; for transgenic Zebrafish see Lin S., Transgenic Zebrafish, Methods Mol. Biol. (2000); 136:375-3830); for microinjection procedures for fish, amphibian eggs and birds see Houdebine and Chourrout, Experientia (1991) 47:897-905; for transgenic rats see Hammer et al., Cell (1990) 63:1099-1112; and for culturing of embryonic stem (ES) cells and the subsequent production of transgenic animals by the introduction of DNA into ES cells using methods such as electroporation, calcium phosphate/DNA precipitation and direct injection see, e.g., Teratocarcinomas and Embryonic Stem Cells, A Practical Approach, E. J. Robertson, ed., IRL Press (1987)). Clones of the nonhuman transgenic animals can be produced according to available methods (see Wilmut, I. et al. (1997) Nature 385:810-813; and PCT International Publication Nos. WO 97/07668 and WO 97/07669).

In one embodiment, the transgenic animal is a “knock-out” animal having a heterozygous or homozygous alteration in the sequence of an endogenous MAP2K6 gene that results in a decrease of MAP2K6 function, preferably such that MAP2K6 expression is undetectable or insignificant. Knock-out animals are typically generated by homologous recombination with a vector comprising a transgene having at least a portion of the gene to be knocked out. Typically a deletion, addition or substitution has been introduced into the transgene to functionally disrupt it. The transgene can be a human gene (e.g., from a human genomic clone) but more preferably is an ortholog of the human gene derived from the transgenic host species. For example, a mouse MAP2K6 gene is used to construct a homologous recombination vector suitable for altering an endogenous MAP2K6 gene in the mouse genome. Detailed methodologies for homologous recombination in mice are available (see Capecchi, Science (1989) 244:1288-1292; Joyner et al., Nature (1989) 338:153-156). Procedures for the production of non-rodent transgenic mammals and other animals are also available (Houdebine and Chourrout, supra; Pursel et al., Science (1989) 244:1281-1288; Simms et al., Bio/Technology (1988) 6:179-183). In a preferred embodiment, knock-out animals, such as mice harboring a knockout of a specific gene, may be used to produce antibodies against the human counterpart of the gene that has been knocked out (Claesson M H et al., (1994) Scan J Immunol 40:257-264; Declerck P J et al., (1995) J Biol Chem. 270:8397-400).

In another embodiment, the transgenic animal is a “knock-in” animal having an alteration in its genome that results in altered expression (e.g., increased (including ectopic) or decreased expression) of the MAP2K6 gene, e.g., by introduction of additional copies of MAP2K6, or by operatively inserting a regulatory sequence that provides for altered expression of an endogenous copy of the MAP2K6 gene. Such regulatory sequences include inducible, tissue-specific, and constitutive promoters and enhancer elements. The knock-in can be homozygous or heterozygous.

Transgenic nonhuman animals can also be produced that contain selected systems allowing for regulated expression of the transgene. One example of such a system that may be produced is the cre/loxP recombinase system of bacteriophage P1 (Lakso et al., PNAS (1992) 89:6232-6236; U.S. Pat. No. 4,959,317). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase. Another example of a recombinase system is the FLP recombinase system of Saccharomyces cerevisiae (O'Gorman et al. (1991) Science 251:1351-1355; U.S. Pat. No. 5,654,182). In a preferred embodiment, both Cre-LoxP and Flp-Frt are used in the same system to regulate expression of the transgene, and for sequential deletion of vector sequences in the same cell (Sun X et al (2000) Nat Genet 25:83-6).

The genetically modified animals can be used in genetic studies to further elucidate branching morphogenesis, as animal models of disease and disorders implicating defective branching morphogenesis function, and for in vivo testing of candidate therapeutic agents, such as those identified in screens described below. The candidate therapeutic agents are administered to a genetically modified animal having altered MAP2K6 function and phenotypic changes are compared with appropriate control animals such as genetically modified animals that receive placebo treatment, and/or animals with unaltered MAP2K6 expression that receive candidate therapeutic agent.

In addition to the above-described genetically modified animals having altered MAP2K6 function, animal models having defective branching morphogenesis function (and otherwise normal MAP2K6 function), can be used in the methods of the present invention. For example, a branching morphogenesis knockout mouse can be used to assess, in vivo, the activity of a candidate branching morphogenesis modulating agent identified in one of the in vitro assays described below. Preferably, the candidate branching morphogenesis modulating agent when administered to a model system with cells defective in branching morphogenesis function, produces a detectable phenotypic change in the model system indicating that the branching morphogenesis function is restored, i.e., the cells exhibit normal branching morphogenesis.

Modulating Agents

The invention provides methods to identify agents that interact with and/or modulate the function of MAP2K6 and/or branching morphogenesis. Modulating agents identified by the methods are also part of the invention. Such agents are useful in a variety of diagnostic and therapeutic applications associated with branching morphogenesis, as well as in further analysis of the MAP2K6 protein and its contribution to branching morphogenesis. Accordingly, the invention also provides methods for modulating branching morphogenesis comprising the step of specifically modulating MAP2K6 activity by administering a MAP2K6-interacting or -modulating agent.

As used herein, a “MAP2K6-modulating agent” is any agent that modulates MAP2K6 function, for example, an agent that interacts with MAP2K6 to inhibit or enhance MAP2K6 activity or otherwise affect normal MAP2K6 function. MAP2K6 function can be affected at any level, including transcription, protein expression, protein localization, and cellular or extra-cellular activity. In a preferred embodiment, the MAP2K6-modulating agent specifically modulates the function of the MAP2K6. The phrases “specific modulating agent”, “specifically modulates”, etc., are used herein to refer to modulating agents that directly bind to the MAP2K6 polypeptide or nucleic acid, and preferably inhibit, enhance, or otherwise alter, the function of the MAP2K6. These phrases also encompass modulating agents that alter the interaction of the MAP2K6 with a binding partner, substrate, or cofactor (e.g. by binding to a binding partner of a MAP2K6, or to a protein/binding partner complex, and altering MAP2K6 function). In a further preferred embodiment, the MAP2K6-modulating agent is a modulator of branching morphogenesis (e.g. it restores and/or upregulates branching morphogenesis function) and thus is also a branching morphogenesis-modulating agent.

Preferred MAP2K6-modulating agents include small molecule compounds; MAP2K6-interacting proteins, including antibodies and other biotherapeutics; and nucleic acid modulators such as antisense and RNA inhibitors. The modulating agents may be formulated in pharmaceutical compositions, for example, as compositions that may comprise other active ingredients, as in combination therapy, and/or suitable carriers or recipients. Techniques for formulation and administration of the compounds may be found in “Remington's Pharmaceutical Sciences” Mack Publishing Co., Easton, Pa., 19^{th} edition.

Small Molecule Modulators

Small molecules are often preferred to modulate function of proteins with enzymatic function, and/or containing protein interaction domains. Chemical agents, referred to in the art as “small molecule” compounds are typically organic, non-peptide molecules, having a molecular weight less than 10,000, preferably less than 5,000, more preferably less than 1,000, and most preferably less than 500 daltons. This class of modulators includes chemically synthesized molecules, for instance, compounds from combinatorial chemical libraries. Synthetic compounds may be rationally designed or identified based on known or inferred properties of the MAP2K6 protein or may be identified by screening compound libraries. Alternative appropriate modulators of this class are natural products, particularly secondary metabolites from organisms such as plants or fungi, which can also be identified by screening compound libraries for MAP2K6-modulating activity. Methods for generating and obtaining compounds are well known in the art (Schreiber S L, Science (2000) 151: 1964-1969; Radmann J and Gunther J, Science (2000) 151:1947-1948).

Small molecule modulators identified from screening assays, as described below, can be used as lead compounds from which candidate clinical compounds may be designed, optimized, and synthesized. Such clinical compounds may have utility in treating pathologies associated with branching morphogenesis. The activity of candidate small molecule modulating agents may be improved several-fold through iterative secondary functional validation, as further described below, structure determination, and candidate modulator modification and testing. Additionally, candidate clinical compounds are generated with specific regard to clinical and pharmacological properties. For example, the reagents may be derivatized and re-screened using in vitro and in vivo assays to optimize activity and minimize toxicity for pharmaceutical development.

Protein Modulators

Specific MAP2K6-interacting proteins are useful in a variety of diagnostic and therapeutic applications related to branching morphogenesis and related disorders, as well as in validation assays for other MAP2K6-modulating agents. In a preferred embodiment, MAP2K6-interacting proteins affect normal MAP2K6 function, including transcription, protein expression, protein localization, and cellular or extra-cellular activity. In another embodiment, MAP2K6-interacting proteins are useful in detecting and providing information about the function of MAP2K6 proteins, as is relevant to branching morphogenesis related disorders, such as cancer (e.g., for diagnostic means).

A MAP2K6-interacting protein may be endogenous, i.e. one that naturally interacts genetically or biochemically with a MAP2K6, such as a member of the MAP2K6 pathway that modulates MAP2K6 expression, localization, and/or activity. MAP2K6-modulators include dominant negative forms of MAP2K6-interacting proteins and of MAP2K6 proteins themselves. Yeast two-hybrid and variant screens offer preferred methods for identifying endogenous MAP2K6-interacting proteins (Finley, R. L. et al. (1996) in DNA Cloning-Expression Systems: A Practical Approach, eds. Glover D. & Hames B. D (Oxford University Press, Oxford, England), pp. 169-203; Fashema S F et al., Gene (2000) 250:1-14; Drees B L Curr Opin Chem Biol (1999) 3:64-70; Vidal M and Legrain P Nucleic Acids Res (1999) 27:919-29; and U.S. Pat. No. 5,928,868). Mass spectrometry is an alternative preferred method for the elucidation of protein complexes (reviewed in, e.g., Pandley A and Mann M, Nature (2000) 405:837-846; Yates J R 3^{rd}, Trends Genet (2000) 16:5-8).

An MAP2K6-interacting protein may be an exogenous protein, such as a MAP2K6-specific antibody or a T-cell antigen receptor (see, e.g., Harlow and Lane (1988) Antibodies, A Laboratory Manual, Cold Spring Harbor Laboratory; Harlow and Lane (1999) Using antibodies: a laboratory manual. Cold Spring Harbor, N.Y.: Cold Spring Harbor Laboratory Press). MAP2K6 antibodies are further discussed below.

In preferred embodiments, a MAP2K6-interacting protein specifically binds a MAP2K6 protein. In alternative preferred embodiments, a MAP2K6-modulating agent binds a MAP2K6 substrate, binding partner, or cofactor.

Antibodies

In another embodiment, the protein modulator is a MAP2K6 specific antibody agonist or antagonist. The antibodies have therapeutic and diagnostic utilities, and can be used in screening assays to identify MAP2K6 modulators. The antibodies can also be used in dissecting the portions of the MAP2K6 pathway responsible for various cellular responses and in the general processing and maturation of the MAP2K6.

Antibodies that specifically bind MAP2K6 polypeptides can be generated using known methods. Preferably the antibody is specific to a mammalian ortholog of MAP2K6 polypeptide, and more preferably, to human MAP2K6. Antibodies may be polyclonal, monoclonal (mAbs), humanized or chimeric antibodies, single chain antibodies, Fab fragments, F(ab′).sub.2 fragments, fragments produced by a FAb expression library, anti-idiotypic (anti-Id) antibodies, and epitope-binding fragments of any of the above. Epitopes of MAP2K6 which are particularly antigenic can be selected, for example, by routine screening of MAP2K6 polypeptides for antigenicity or by applying a theoretical method for selecting antigenic regions of a protein (Hopp and Wood (1981), Proc. Natl. Acad. Sci. U.S.A. 78:3824-28; Hopp and Wood, (1983) Mol. Immunol. 20:483-89; Sutcliffe et al., (1983) Science 219:660-66) to the amino acid sequence of MAP2K6. Monoclonal antibodies with affinities of 10⁸ M⁻¹ preferably 10⁹ M⁻¹ to 10¹⁰ M⁻¹, or stronger can be made by standard procedures as described (Harlow and Lane, supra; Goding (1986) Monoclonal Antibodies: Principles and Practice (2d ed) Academic Press, New York; and U.S. Pat. Nos. 4,381,292; 4,451,570; and 4,618,577). Antibodies may be generated against crude cell extracts of MAP2K6 or substantially purified fragments thereof. If MAP2K6 fragments are used, they preferably comprise at least 10, and more preferably, at least 20 contiguous amino acids of a MAP2K6 protein. In a particular embodiment, MAP2K6-specific antigens and/or immunogens are coupled to carrier proteins that stimulate the immune response. For example, the subject polypeptides are covalently coupled to the keyhole limpet hemocyanin (KLH) carrier, and the conjugate is emulsified in Freund's complete adjuvant, which enhances the immune response. An appropriate immune system such as a laboratory rabbit or mouse is immunized according to conventional protocols.

The presence of MAP2K6-specific antibodies is assayed by an appropriate assay such as a solid phase enzyme-linked immunosorbant assay (ELISA) using immobilized corresponding MAP2K6 polypeptides. Other assays, such as radioimmunoassays or fluorescent assays might also be used.

Chimeric antibodies specific to MAP2K6 polypeptides can be made that contain different portions from different animal species. For instance, a human immunoglobulin constant region may be linked to a variable region of a murine mAb, such that the antibody derives its biological activity from the human antibody, and its binding specificity from the murine fragment. Chimeric antibodies are produced by splicing together genes that encode the appropriate regions from each species (Morrison et al., Proc. Natl. Acad. Sci. (1984) 81:6851-6855; Neuberger et al., Nature (1984) 312:604-608; Takeda et al., Nature (1985) 31:452-454). Humanized antibodies, which are a form of chimeric antibodies, can be generated by grafting complementary-determining regions (CDRs) (Carlos, T. M., J. M. Harlan. 1994. Blood 84:2068-2101) of mouse antibodies into a background of human framework regions and constant regions by recombinant DNA technology (Riechmann L M, et al., 1988 Nature 323: 323-327). Humanized antibodies contain ˜10% murine sequences and ˜90% human sequences, and thus further reduce or eliminate immunogenicity, while retaining the antibody specificities (Co M S, and Queen C. 1991 Nature 351: 501-501; Morrison S L. 1992 Ann. Rev. Immun. 10:239-265). Humanized antibodies and methods of their production are well-known in the art (U.S. Pat. Nos. 5,530,101, 5,585,089, 5,693,762, and 6,180,370).

MAP2K6-specific single chain antibodies which are recombinant, single chain polypeptides formed by linking the heavy and light chain fragments of the Fv regions via an amino acid bridge, can be produced by methods known in the art (U.S. Pat. No. 4,946,778; Bird, Science (1988) 242:423-426; Huston et al., Proc. Natl. Acad. Sci. USA (1988) 85:5879-5883; and Ward et al., Nature (1989) 334:544-546).

Other suitable techniques for antibody production involve in vitro exposure of lymphocytes to the antigenic polypeptides or alternatively to selection of libraries of antibodies in phage or similar vectors (Huse et al., Science (1989) 246:1275-1281). As used herein, T-cell antigen receptors are included within the scope of antibody modulators (Harlow and Lane, 1988, supra).

The polypeptides and antibodies of the present invention may be used with or without modification. Frequently, antibodies will be labeled by joining, either covalently or non-covalently, a substance that provides for a detectable signal, or that is toxic to cells that express the targeted protein (Menard S, et al., Int J. Biol Markers (1989) 4:131-134). A wide variety of labels and conjugation techniques are known and are reported extensively in both the scientific and patent literature. Suitable labels include radionuclides, enzymes, substrates, cofactors, inhibitors, fluorescent moieties, fluorescent emitting lanthanide metals, chemiluminescent moieties, bioluminescent moieties, magnetic particles, and the like (U.S. Pat. Nos. 3,817,837; 3,850,752; 3,939,350; 3,996,345; 4,277,437; 4,275,149; and 4,366,241). Also, recombinant immunoglobulins may be produced (U.S. Pat. No. 4,816,567). Antibodies to cytoplasmic polypeptides may be delivered and reach their targets by conjugation with membrane-penetrating toxin proteins (U.S. Pat. No. 6,086,900).

When used therapeutically in a patient, the antibodies of the subject invention are typically administered parenterally, when possible at the target site, or intravenously. The therapeutically effective dose and dosage regimen is determined by clinical studies. Typically, the amount of antibody administered is in the range of about 0.1 mg/kg-to about 10 mg/kg of patient weight. For parenteral administration, the antibodies are formulated in a unit dosage injectable form (e.g., solution, suspension, emulsion) in association with a pharmaceutically acceptable vehicle. Such vehicles are inherently nontoxic and non-therapeutic. Examples are water, saline, Ringer's solution, dextrose solution, and 5% human serum albumin. Nonaqueous vehicles such as fixed oils, ethyl oleate, or liposome carriers may also be used. The vehicle may contain minor amounts of additives, such as buffers and preservatives, which enhance isotonicity and chemical stability or otherwise enhance therapeutic potential. The antibodies' concentrations in such vehicles are typically in the range of about 1 mg/ml to about 10 mg/ml. Immunotherapeutic methods are further described in the literature (U.S. Pat. No. 5,859,206; WO0073469).

Nucleic Acid Modulators

Other preferred MAP2K6-modulating agents comprise nucleic acid molecules, such as antisense oligomers or double stranded RNA (dsRNA), which generally inhibit MAP2K6 activity. Preferred nucleic acid modulators interfere with the function of the MAP2K6 nucleic acid such as DNA replication, transcription, translocation of the MAP2K6 RNA to the site of protein translation, translation of protein from the MAP2K6 RNA, splicing of the MAP2K6 RNA to yield one or more mRNA species, or catalytic activity which may be engaged in or facilitated by the MAP2K6 RNA.

In one embodiment, the antisense oligomer is an oligonucleotide that is sufficiently complementary to a MAP2K6 mRNA to bind to and prevent translation, preferably by binding to the 5′ untranslated region. MAP2K6-specific antisense oligonucleotides, preferably range from at least 6 to about 200 nucleotides. In some embodiments the oligonucleotide is preferably at least 10, 15, or 20 nucleotides in length. In other embodiments, the oligonucleotide is preferably less than 50, 40, or 30 nucleotides in length. The oligonucleotide can be DNA or RNA or a chimeric mixture or derivatives or modified versions thereof, single-stranded or double-stranded. The oligonucleotide can be modified at the base moiety, sugar moiety, or phosphate backbone. The oligonucleotide may include other appending groups such as peptides, agents that facilitate transport across the cell membrane, hybridization-triggered cleavage agents, and intercalating agents.

In another embodiment, the antisense oligomer is a phosphothioate morpholino oligomer (PMO). PMOs are assembled from four different morpholino subunits, each of which contain one of four genetic bases (A, C, G, or T) linked to a six-membered morpholine ring. Polymers of these subunits are joined by non-ionic phosphodiamidate intersubunit linkages. Details of how to make and use PMOs and other antisense oligomers are well known in the art (e.g. see WO99/18193; Probst J C, Antisense Oligodeoxynucleotide and Ribozyme Design, Methods. (2000) 22(3):271-281; Summerton J, and Weller D. 1997 Antisense Nucleic Acid Drug Dev.:7:187-95; U.S. Pat. No. 5,235,033; and U.S. Pat. No. 5,378,841).

Alternative preferred MAP2K6 nucleic acid modulators are double-stranded RNA species mediating RNA interference (RNAi). RNAi is the process of sequence-specific, post-transcriptional gene silencing in animals and plants, initiated by double-stranded RNA (dsRNA) that is homologous in sequence to the silenced gene. Methods relating to the use of RNAi to silence genes in C. elegans, Drosophila, plants, and humans are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619; Elbashir S M, et al., 2001 Nature 411:494-498).

Nucleic acid modulators are commonly used as research reagents, diagnostics, and therapeutics. For example, antisense oligonucleotides, which are able to inhibit gene expression with exquisite specificity, are often used to elucidate the function of particular genes (see, for example, U.S. Pat. No. 6,165,790). Nucleic acid modulators are also used, for example, to distinguish between functions of various members of a biological pathway. For example, antisense oligomers have been employed as therapeutic moieties in the treatment of disease states in animals and man and have been demonstrated in numerous clinical trials to be safe and effective (Milligan J F, et al, Current Concepts in Antisense Drug Design, J Med Chem. (1993) 36:1923-1937; Tonkinson J L et al., Antisense Oligodeoxynucleotides as Clinical Therapeutic Agents, Cancer Invest. (1996) 14:54-65). Accordingly, in one aspect of the invention, a MAP2K6-specific nucleic acid modulator is used in an assay to further elucidate the role of the MAP2K6 in branching morphogenesis, [symbol] its relationship to other members of the pathway. In another aspect of the invention, a MAP2K6-specific antisense oligomer is used as a therapeutic agent for treatment of branching morphogenesis-related disease states.

Zebrafish is a particularly useful model for the study of branching morphogenesis using antisense oligomers. For example, PMOs are used to selectively inactive one or more genes in vivo in the Zebrafish embryo. By injecting PMOs into Zebrafish at the 1-16 cell stage candidate targets emerging from the Drosophila screens are validated in this vertebrate model system. In another aspect of the invention, PMOs are used to screen the Zebrafish genome for identification of other therapeutic modulators of branching morphogenesis. In a further aspect of the invention, a MAP2K6-specific antisense oligomer is used as a therapeutic agent for treatment of pathologies associated with branching morphogenesis.

Assay Systems

The invention provides assay systems and screening methods for identifying specific modulators of MAP2K6 activity. As used herein, an “assay system” encompasses all the components required for performing and analyzing results of an assay that detects and/or measures a particular event. In general, primary assays are used to identify or confirm a modulator's specific biochemical or molecular effect with respect to the MAP2K6 nucleic acid or protein. In general, secondary assays further assess the activity of a MAP2K6 modulating agent identified by a primary assay and may confirm that the modulating agent affects MAP2K6 in a manner relevant to branching morphogenesis. In some cases, MAP2K6 modulators will be directly tested in a secondary assay.

In a preferred embodiment, the screening method comprises contacting a suitable assay system comprising a MAP2K6 polypeptide or nucleic acid with a candidate agent under conditions whereby, but for the presence of the agent, the system provides a reference activity (e.g. kinase activity), which is based on the particular molecular event the screening method detects. A statistically significant difference between the agent-biased activity and the reference activity indicates that the candidate agent modulates MAP2K6 activity, and hence branching morphogenesis. The MAP2K6 polypeptide or nucleic acid used in the assay may comprise any of the nucleic acids or polypeptides described above.

Primary Assays

The type of modulator tested generally determines the type of primary assay.

Primary Assays for Small Molecule Modulators

For small molecule modulators, screening assays are used to identify candidate modulators. Screening assays may be cell-based or may use a cell-free system that recreates or retains the relevant biochemical reaction of the target protein (reviewed in Sittampalam G S et al., Curr Opin Chem Biol (1997) 1:384-91 and accompanying references). As used herein the term “cell-based” refers to assays using live cells, dead cells, or a particular cellular fraction, such as a membrane, endoplasmic reticulum, or mitochondrial fraction. The term “cell free” encompasses assays using substantially purified protein (either endogenous or recombinantly produced), partially purified or crude cellular extracts. Screening assays may detect a variety of molecular events, including protein-DNA interactions, protein-protein interactions (e.g., receptor-ligand binding), transcriptional activity (e.g., using a reporter gene), enzymatic activity (e.g., via a property of the substrate), activity of second messengers, immunogenicity and changes in cellular morphology or other cellular characteristics. Appropriate screening assays may use a wide range of detection methods including fluorescent, radioactive, calorimetric, spectrophotometric, and amperometric methods, to provide a read-out for the particular molecular event detected.

Cell-based screening assays usually require systems for recombinant expression of MAP2K6 and any auxiliary proteins demanded by the particular assay. Appropriate methods for generating recombinant proteins produce sufficient quantities of proteins that retain their relevant biological activities and are of sufficient purity to optimize activity and assure assay reproducibility. Yeast two-hybrid and variant screens, and mass spectrometry provide preferred methods for determining protein-protein interactions and elucidation of protein complexes. In certain applications, when MAP2K6-interacting proteins are used in screens to identify small molecule modulators, the binding specificity of the interacting protein to the MAP2K6 protein may be assayed by various known methods such as substrate processing (e.g. ability of the candidate MAP2K6-specific binding agents to function as negative effectors in MAP2K6-expressing cells), binding equilibrium constants (usually at least about 10⁷ M⁻¹, preferably at least about 10⁸ M⁻¹, more preferably at least about 10⁹ M⁻¹), and immunogenicity (e.g. ability to elicit MAP2K6 specific antibody in a heterologous host such as a mouse, rat, goat or rabbit). For enzymes and receptors, binding may be assayed by, respectively, substrate and ligand processing.

The screening assay may measure a candidate agent's ability to specifically bind to or modulate activity of a MAP2K6 polypeptide, a fusion protein thereof, or to cells or membranes bearing the polypeptide or fusion protein. The MAP2K6 polypeptide can be full length or a fragment thereof that retains functional MAP2K6 activity. The MAP2K6 polypeptide may be fused to another polypeptide, such as a peptide tag for detection or anchoring, or to another tag. The MAP2K6 polypeptide is preferably human MAP2K6, or is an ortholog or derivative thereof as described above. In a preferred embodiment, the screening assay detects candidate agent-based modulation of MAP2K6 interaction with a binding target, such as an endogenous or exogenous protein or other substrate that has MAP2K6-specific binding activity, and can be used to assess normal MAP2K6 gene function.

Suitable assay formats that may be adapted to screen for MAP2K6 modulators are known in the art. Preferred screening assays are high throughput or ultra high throughput and thus provide automated, cost-effective means of screening compound libraries for lead compounds (Fernandes P B, Curr Opin Chem Biol (1998) 2:597-603; Sundberg S A, Curr Opin Biotechnol 2000, 11:47-53). In one preferred embodiment, screening assays uses fluorescence technologies, including fluorescence polarization, time-resolved fluorescence, and fluorescence resonance energy transfer. These systems offer means to monitor protein-protein or DNA-protein interactions in which the intensity of the signal emitted from dye-labeled molecules depends upon their interactions with partner molecules (e.g., Selvin P R, Nat Struct Biol (2000) 7:730-4; Fernandes P B, supra; Hertzberg R P and Pope A J, Curr Opin Chem Biol (2000) 4:445-451).

A variety of suitable assay systems may be used to identify candidate MAP2K6 and branching morphogenesis modulators (e.g. U.S. Pat. No. 6,165,992 (kinase assays); U.S. Pat. Nos. 5,550,019 and 6,133,437 (apoptosis assays); and U.S. Pat. Nos. 5,976,782, 6,225,118 and 6,444,434 (angiogenesis assays), among others). Specific preferred assays are described in more detail below.

Kinase assays. In some preferred embodiments the screening assay detects the ability of the test agent to modulate the kinase activity of a MAP2K6 polypeptide. In further embodiments, a cell-free kinase assay system is used to identify a candidate branching morphogenesis modulating agent, and a secondary, cell-based assay, such as an apoptosis or hypoxic induction assay (described below), may be used to further characterize the candidate branching morphogenesis modulating agent. Many different assays for kinases have been reported in the literature and are well known to those skilled in the art (e.g. U.S. Pat. No. 6,165,992; Zhu et al., Nature Genetics (2000) 26:283-289; and WO0073469). Radioassays, which monitor the transfer of a gamma phosphate are frequently used. For instance, a scintillation assay for p56 (lck) kinase activity monitors the transfer of the gamma phosphate from gamma-³³P ATP to a biotinylated peptide substrate; the substrate is captured on a streptavidin coated bead that transmits the signal (Beveridge M et al., J Biomol Screen (2000) 5:205-212). This assay uses the scintillation proximity assay (SPA), in which only radio-ligand bound to receptors tethered to the surface of an SPA bead are detected by the scintillant immobilized within it, allowing binding to be measured without separation of bound from free ligand.

Other assays for protein kinase activity may use antibodies that specifically recognize phosphorylated substrates. For instance, the kinase receptor activation (KIRA) assay measures receptor tyrosine kinase activity by ligand stimulating the intact receptor in cultured cells, then capturing solubilized receptor with specific antibodies and quantifying phosphorylation via phosphotyrosine ELISA (Sadick M D, Dev Biol Stand (1999) 97:121-133).

Another example of antibody based assays for protein kinase activity is TRF (time-resolved fluorometry). This method utilizes europium chelate-labeled anti-phosphotyrosine antibodies to detect phosphate transfer to a polymeric substrate coated onto microtiter plate wells. The amount of phosphorylation is then detected using time-resolved, dissociation-enhanced fluorescence (Braunwalder A F, et al., Anal Biochem 1996 Jul. 1; 238(2):159-64).

Apoptosis assays. Assays for apoptosis may be performed by terminal deoxynucleotidyl transferase-mediated digoxigenin-11-dUTP nick end labeling (TUNEL) assay. The TUNEL assay is used to measure nuclear DNA fragmentation characteristic of apoptosis (Lazebnik et al., 1994, Nature 371, 346), by following the incorporation of fluorescein-dUTP (Yonehara et al., 1989, J. Exp. Med. 169, 1747). Apoptosis may further be assayed by acridine orange staining of tissue culture cells (Lucas, R., et al., 1998, Blood 15:4730-41). Other cell-based apoptosis assays include the caspase-3/7 assay and the cell death nucleosome ELISA assay. The caspase 3/7 assay is based on the activation of the caspase cleavage activity as part of a cascade of events that occur during programmed cell death in many apoptotic pathways. In the caspase 3/7 assay (commercially available Apo-ONE™ Homogeneous Caspase-3/7 assay from Promega, cat# 67790), lysis buffer and caspase substrate are mixed and added to cells. The caspase substrate becomes fluorescent when cleaved by active caspase 3/7. The nucleosome ELISA assay is a general cell death assay known to those skilled in the art, and available commercially (Roche, Cat# 1774425). This assay is a quantitative sandwich-enzyme-immunoassay which uses monoclonal antibodies directed against DNA and histones respectively, thus specifically determining amount of mono- and oligonucleosomes in the cytoplasmic fraction of cell lysates. Mono and oligonucleosomes are enriched in the cytoplasm during apoptosis due to the fact that DNA fragmentation occurs several hours before the plasma membrane breaks down, allowing for accumulation in the cytoplasm. Nucleosomes are not present in the cytoplasmic fraction of cells that are not undergoing apoptosis. An apoptosis assay system may comprise a cell that expresses a MAP2K6, and that optionally has defective branching morphogenesis function. A test agent can be added to the apoptosis assay system and changes in induction of apoptosis relative to controls where no test agent is added, identify candidate branching morphogenesis modulating agents. In some embodiments of the invention, an apoptosis assay may be used as a secondary assay to test a candidate branching morphogenesis modulating agents that is initially identified using a cell-free assay system. An apoptosis assay may also be used to test whether MAP2K6 function plays a direct role in apoptosis. For example, an apoptosis assay may be performed on cells that over- or under-express MAP2K6 relative to wild type cells. Differences in apoptotic response compared to wild type cells suggests that the MAP2K6 plays a direct role in the apoptotic response. Apoptosis assays are described further in U.S. Pat. No. 6,133,437.

Cell proliferation and cell cycle assays. Cell proliferation may be assayed via bromodeoxyuridine (BRDU) incorporation. This assay identifies a cell population undergoing DNA synthesis by incorporation of BRDU into newly-synthesized DNA. Newly-synthesized DNA may then be detected using an anti-BRDU antibody (Hoshino et al., 1986, Int. J. Cancer 38, 369; Campana et al., 1988, J. Immunol. Meth. 107, 79), or by other means.

Cell proliferation is also assayed via phospho-histone H3 staining, which identifies a cell population undergoing mitosis by phosphorylation of histone H3. Phosphorylation of histone H3 at serine 10 is detected using an antibody specific to the phosphorylated form of the serine 10 residue of histone H3. (Chadlee, D. N. 1995, J. Biol. Chem 270:20098-105). Cell Proliferation may also be examined using [³H]-thymidine incorporation (Chen, J., 1996, Oncogene 13:1395-403; Jeoung, J., 1995, J. Biol. Chem. 270:18367-73). This assay allows for quantitative characterization of S-phase DNA syntheses. In this assay, cells synthesizing DNA will incorporate [³H]-thymidine into newly synthesized DNA. Incorporation can then be measured by standard techniques such as by counting of radioisotope in a scintillation counter (e.g., Beckman L S 3800 Liquid Scintillation Counter). Another proliferation assay uses the dye Alamar Blue (available from Biosource International), which fluoresces when reduced in living cells and provides an indirect measurement of cell number (Voytik-Harbin S L et al., 1998, In Vitro Cell Dev Biol Anim 34:239-46). Yet another proliferation assay, the MTS assay, is based on in vitro cytotoxicity assessment of industrial chemicals, and uses the soluble tetrazolium salt, MTS. MTS assays are commercially available, for example, the Promega CellTiter 96® AQueous Non-Radioactive Cell Proliferation Assay (Cat.# G5421).

Cell proliferation may also be assayed by colony formation in soft agar (Sambrook et al., Molecular Cloning, Cold Spring Harbor (1989)). For example, cells transformed with MAP2K6 are seeded in soft agar plates, and colonies are measured and counted after two weeks incubation.

Cell proliferation may also be assayed by measuring ATP levels as indicator of metabolically active cells. Such assays are commercially available, for example Cell Titer-Glo™, which is a luminescent homogeneous assay available from Promega.

Involvement of a gene in the cell cycle may be assayed by flow cytometry (Gray J W et al. (1986) Int J Radiat Biol Relat Stud Phys Chem Med 49:237-55). Cells transfected with a MAP2K6 may be stained with propidium iodide and evaluated in a flow cytometer (available from Becton Dickinson), which indicates accumulation of cells in different stages of the cell cycle.

Accordingly, a cell proliferation or cell cycle assay system may comprise a cell that expresses a MAP2K6, and that optionally has defective branching morphogenesis function. A test agent can be added to the assay system and changes in cell proliferation or cell cycle relative to controls where no test agent is added, identify candidate branching morphogenesis modulating agents. In some embodiments of the invention, the cell proliferation or cell cycle assay may be used as a secondary assay to test a candidate branching morphogenesis modulating agents that is initially identified using another assay system such as a cell-free assay system. A cell proliferation assay may also be used to test whether MAP2K6 function plays a direct role in cell proliferation or cell cycle. For example, a cell proliferation or cell cycle assay may be performed on cells that over- or under-express MAP2K6 relative to wild type cells. Differences in proliferation or cell cycle compared to wild type cells suggests that the MAP2K6 plays a direct role in cell proliferation or cell cycle.

Angiogenesis. Angiogenesis may be assayed using various human endothelial cell systems, such as umbilical vein, coronary artery, or dermal cells. Suitable assays include Alamar Blue based assays (available from Biosource International) to measure proliferation; migration assays using fluorescent molecules, such as the use of Becton Dickinson Falcon HTS FluoroBlock cell culture inserts to measure migration of cells through membranes in presence or absence of angiogenesis enhancer or suppressors; and tubule formation assays based on the formation of tubular structures by endothelial cells on Matrigel® (Becton Dickinson). Accordingly, an angiogenesis assay system may comprise a cell that expresses a MAP2K6, and that optionally has defective branching morphogenesis function. A test agent can be added to the angiogenesis assay system and changes in angiogenesis relative to controls where no test agent is added, identify candidate branching morphogenesis modulating agents. In some embodiments of the invention, the angiogenesis assay may be used as a secondary assay to test a candidate branching morphogenesis modulating agents that is initially identified using another assay system. An angiogenesis assay may also be used to test whether MAP2K6 function plays a direct role in cell proliferation. For example, an angiogenesis assay may be performed on cells that over- or under-express MAP2K6 relative to wild type cells. Differences in angiogenesis compared to wild type cells suggests that the MAP2K6 plays a direct role in angiogenesis. U.S. Pat. Nos. 5,976,782, 6,225,118 and 6,444,434, among others, describe various angiogenesis assays.

Hypoxic induction. The alpha subunit of the transcription factor, hypoxia inducible factor-1 (HIF-1), is upregulated in tumor cells following exposure to hypoxia in vitro. Under hypoxic conditions, HIF-1 stimulates the expression of genes known to be important in tumour cell survival, such as those encoding glyolytic enzymes and VEGF. Induction of such genes by hypoxic conditions may be assayed by growing cells transfected with MAP2K6 in hypoxic conditions (such as with 0.1% O2, 5% CO2, and balance N2, generated in a Napco 7001 incubator (Precision Scientific)) and normoxic conditions, followed by assessment of gene activity or expression by Taqman®. For example, a hypoxic induction assay system may comprise a cell that expresses a MAP2K6, and that optionally has defective branching morphogenesis function. A test agent can be added to the hypoxic induction assay system and changes in hypoxic response relative to controls where no test agent is added, identify candidate branching morphogenesis modulating agents. In some embodiments of the invention, the hypoxic induction assay may be used as a secondary assay to test a candidate branching morphogenesis modulating agents that is initially identified using another assay system. A hypoxic induction assay may also be used to test whether MAP2K6 function plays a direct role in the hypoxic response. For example, a hypoxic induction assay may be performed on cells that over- or under-express MAP2K6 relative to wild type cells. Differences in hypoxic response compared to wild type cells suggests that the MAP2K6 plays a direct role in hypoxic induction.

Cell adhesion. Cell adhesion assays measure adhesion of cells to purified adhesion proteins, or adhesion of cells to each other, in presence or absence of candidate modulating agents. Cell-protein adhesion assays measure the ability of agents to modulate the adhesion of cells to purified proteins. For example, recombinant proteins are produced, diluted to 2.5 g/mL in PBS, and used to coat the wells of a microtiter plate. The wells used for negative control are not coated. Coated wells are then washed, blocked with 1% BSA, and washed again. Compounds are diluted to 2× final test concentration and added to the blocked, coated wells. Cells are then added to the wells, and the unbound cells are washed off. Retained cells are labeled directly on the plate by adding a membrane-permeable fluorescent dye, such as calcein-AM, and the signal is quantified in a fluorescent microplate reader.

Cell-cell adhesion assays measure the ability of agents to modulate binding of cell adhesion proteins with their native ligands. These assays use cells that naturally or recombinantly express the adhesion protein of choice. In an exemplary assay, cells expressing the cell adhesion protein are plated in wells of a multiwell plate. Cells expressing the ligand are labeled with a membrane-permeable fluorescent dye, such as BCECF, and allowed to adhere to the monolayers in the presence of candidate agents. Unbound cells are washed off, and bound cells are detected using a fluorescence plate reader.

High-throughput cell adhesion assays have also been described. In one such assay, small molecule ligands and peptides are bound to the surface of microscope slides using a microarray spotter, intact cells are then contacted with the slides, and unbound cells are washed off. In this assay, not only the binding specificity of the peptides and modulators against cell lines are determined, but also the functional cell signaling of attached cells using immunofluorescence techniques in situ on the microchip is measured (Falsey J R et al., Bioconjug Chem. 2001 May-June; 12(3):346-53).

Tubulogenesis. Tubulogenesis assays monitor the ability of cultured cells, generally endothelial cells, to form tubular structures on a matrix substrate, which generally simulates the environment of the extracellular matrix. Exemplary substrates include Matrigel™ (Becton Dickinson), an extract of basement membrane proteins containing laminin, collagen IV, and heparin sulfate proteoglycan, which is liquid at 4° C. and forms a solid gel at 37° C. Other suitable matrices comprise extracellular components such as collagen, fibronectin, and/or fibrin. Cells are stimulated with a pro-angiogenic stimulant, and their ability to form tubules is detected by imaging. Tubules can generally be detected after an overnight incubation with stimuli, but longer or shorter time frames may also be used. Tube formation assays are well known in the art (e.g., Jones M K et al., 1999, Nature Medicine 5:1418-1423). These assays have traditionally involved stimulation with serum or with the growth factors FGF or VEGF. Serum represents an undefined source of growth factors. In a preferred embodiment, the assay is performed with cells cultured in serum free medium, in order to control which process or pathway a candidate agent modulates. Moreover, we have found that different target genes respond differently to stimulation with different pro-angiogenic agents, including inflammatory angiogenic factors such as TNF-alpa. Thus, in a further preferred embodiment, a tubulogenesis assay system comprises testing a MAP2K6's response to a variety of factors, such as FGF, VEGF, phorbol myristate acetate (PMA), TNF-alpha, ephrin, etc.

Cell Migration. An invasion/migration assay (also called a migration assay) tests the ability of cells to overcome a physical barrier and to migrate towards pro-angiogenic signals. Migration assays are known in the art (e.g., Paik J H et al., 2001, J Biol Chem 276:11830-11837). In a typical experimental set-up, cultured endothelial cells are seeded onto a matrix-coated porous lamina, with pore sizes generally smaller than typical cell size. The matrix generally simulates the environment of the extracellular matrix, as described above. The lamina is typically a membrane, such as the transwell polycarbonate membrane (Corning Costar Corporation, Cambridge, Mass.), and is generally part of an upper chamber that is in fluid contact with a lower chamber containing pro-angiogenic stimuli. Migration is generally assayed after an overnight incubation with stimuli, but longer or shorter time frames may also be used. Migration is assessed as the number of cells that crossed the lamina, and may be detected by staining cells with hemotoxylin solution (VWR Scientific, South San Francisco, Calif.), or by any other method for determining cell number. In another exemplary set up, cells are fluorescently labeled and migration is detected using fluorescent readings, for instance using the Falcon HTS FluoroBlok (Becton Dickinson). While some migration is observed in the absence of stimulus, migration is greatly increased in response to pro-angiogenic factors. As described above, a preferred assay system for migration/invasion assays comprises testing a MAP2K6's response to a variety of pro-angiogenic factors, including tumor angiogenic and inflammatory angiogenic agents, and culturing the cells in serum free medium.

Sprouting assay. A sprouting assay is a three-dimensional in vitro angiogenesis assay that uses a cell-number defined spheroid aggregation of endothelial cells (“spheroid”), embedded in a collagen gel-based matrix. The spheroid can serve as a starting point for the sprouting of capillary-like structures by invasion into the extracellular matrix (termed “cell sprouting”) and the subsequent formation of complex anastomosing networks (Korff and Augustin, 1999, J Cell Sci 112:3249-58). In an exemplary experimental set-up, spheroids are prepared by pipetting 400 human umbilical vein endothelial cells into individual wells of a nonadhesive 96-well plates to allow overnight spheroidal aggregation (Korff and Augustin: J Cell Biol 143: 1341-52, 1998). Spheroids are harvested and seeded in 900 μl of methocel-collagen solution and pipetted into individual wells of a 24 well plate to allow collagen gel polymerization. Test agents are added after 30 min by pipetting 100 μl of 10-fold concentrated working dilution of the test substances on top of the gel. Plates are incubated at 37° C. for 24 h. Dishes are fixed at the end of the experimental incubation period by addition of paraformaldehyde. Sprouting intensity of endothelial cells can be quantitated by an automated image analysis system to determine the cumulative sprout length per spheroid.

Primary Assays for Antibody Modulators

For antibody modulators, appropriate primary assays test is a binding assay that tests the antibody's affinity to and specificity for the MAP2K6 protein. Methods for testing antibody affinity and specificity are well known in the art (Harlow and Lane, 1988, 1999, supra). The enzyme-linked immunosorbant assay (ELISA) is a preferred method for detecting MAP2K6-specific antibodies; others include FACS assays, radioimmunoassays, and fluorescent assays.

In some cases, screening assays described for small molecule modulators may also be used to test antibody modulators.

Primary Assays for Nucleic Acid Modulators

For nucleic acid modulators, primary assays may test the ability of the nucleic acid modulator to inhibit or enhance MAP2K6 gene expression, preferably mRNA expression. In general, expression analysis comprises comparing MAP2K6 expression in like populations of cells (e.g., two pools of cells that endogenously or recombinantly express MAP2K6) in the presence and absence of the nucleic acid modulator. Methods for analyzing mRNA and protein expression are well known in the art. For instance, Northern blotting, slot blotting, ribonuclease protection, quantitative RT-PCR (e.g., using the TaqMan®, PE Applied Biosystems), or microarray analysis may be used to confirm that MAP2K6 mRNA expression is reduced in cells treated with the nucleic acid modulator (e.g., Current Protocols in Molecular Biology (1994) Ausubel F M et al., eds., John Wiley & Sons, Inc., chapter 4; Freeman W M et al., Biotechniques (1999) 26:112-125; Kallioniemi O P, Ann Med 2001, 33:142-147; Blohm D H and Guiseppi-Elie, A Curr Opin Biotechnol 2001, 12:41-47). Protein expression may also be monitored. Proteins are most commonly detected with specific antibodies or antisera directed against either the MAP2K6 protein or specific peptides. A variety of means including Western blotting, ELISA, or in situ detection, are available (Harlow E and Lane D, 1988 and 1999, supra).

In some cases, screening assays described for small molecule modulators, particularly in assay systems that involve MAP2K6 mRNA expression, may also be used to test nucleic acid modulators.

Secondary Assays

Secondary assays may be used to further assess the activity of MAP2K6-modulating agent identified by any of the above methods to confirm that the modulating agent affects MAP2K6 in a manner relevant to branching morphogenesis. As used herein, MAP2K6-modulating agents encompass candidate clinical compounds or other agents derived from previously identified modulating agent. Secondary assays can also be used to test the activity of a modulating agent on a particular genetic or biochemical pathway or to test the specificity of the modulating agent's interaction with MAP2K6.

Secondary assays generally compare like populations of cells or animals (e.g., two pools of cells or animals that endogenously or recombinantly express MAP2K6) in the presence and absence of the candidate modulator. In general, such assays test whether treatment of cells or animals with a candidate MAP2K6-modulating agent results in changes in branching morphogenesis in comparison to untreated (or mock- or placebo-treated) cells or animals. Certain assays use “sensitized genetic backgrounds”, which, as used herein, describe cells or animals engineered for altered expression of genes in the branching morphogenesis or interacting pathways.

Cell-Based Assays

Cell based assays may use a variety of mammalian cell types. Preferred cells are capable of branching morphogenesis processes and are generally endothelial cells. Exemplary cells include human umbilical vein endothelial cells (HUVECs), human renal microvascular endothelial cells (HRMECs), human dermal microvascular endothelial cells (HDMECs), human uterine microvascular endothelial cells, human lung microvascular endothelial cells, human coronary artery endothelial cells, and immortalized microvascular cells, among others. Cell based assays may rely on the endogenous expression of MAP2K6 and/or other genes, such as those involved in branching morphogenesis, or may involve recombinant expression of these genes. Candidate modulators are typically added to the cell media but may also be injected into cells or delivered by any other efficacious means.

Cell-based assays may detect a variety of events associated with branching morphogenesis and angiogenesis, including cell proliferation, apoptosis, cell migration, tube formation, sprouting and hypoxic induction, as described above.

Animal Assays

A variety of non-human animal models of branching morphogenesis, including angiogenesis, and related pathologies may be used to test candidate MAP2K6 modulators. Animal assays may rely on the endogenous expression of MAP2K6 and/or other genes, such as those involved in branching morphogenesis, or may involve engineered expression of these genes. In some cases, MAP2K6 expression or MAP2K6 protein may be restricted to a particular implanted tissue or matrix. Animal assays generally require systemic delivery of a candidate modulator, such as by oral administration, injection (intravenous, subcutaneous, intraperitoneous), bolus administration, etc.

In a preferred embodiment, branching morphogenesis activity is assessed by monitoring neovascularization and angiogenesis. Animal models with defective and normal branching morphogenesis are used to test the candidate modulator's affect on MAP2K6 in Matrigel® assays. Matrigel® is an extract of basement membrane proteins, and is composed primarily of laminin, collagen IV, and heparin sulfate proteoglycan. It is provided as a sterile liquid at 4° C., but rapidly forms a solid gel at 37° C. Liquid Matrigel® is mixed with various angiogenic agents, such as bFGF and VEGF, or with human tumor cells which over-express the MAP2K6. The mixture is then injected subcutaneously (SC) into female athymic nude mice (Taconic, Germantown, N.Y.) to support an intense vascular response. Mice with Matrigel® pellets may be dosed via oral (PO), intraperitoneal (IP), or intravenous (IV) routes with the candidate modulator. Mice are euthanized 5-12 days post-injection, and the Matrigel® pellet is harvested for hemoglobin analysis (Sigma plasma hemoglobin kit). Hemoglobin content of the gel is found to correlate the degree of neovascularization in the gel.

In another preferred embodiment, the effect of the candidate modulator on MAP2K6 is assessed via tumorigenicity assays. In one example, a xenograft comprising human cells from a pre-existing tumor or a tumor cell line known to be angiogenic is used; exemplary cell lines include A431, Colo205, MDA-MB-435, A673, A375, Calu-6, MDA-MB-231, 460, SF763T, or SKOV3tp5. Tumor xenograft assays are known in the art (see, e.g., Ogawa K et al., 2000, Oncogene 19:6043-6052). Xenografts are typically implanted SC into female athymic mice, 6-7 week old, as single cell suspensions either from a pre-existing tumor or from in vitro culture. The tumors which express the MAP2K6 endogenously are injected in the flank, 1×10⁵ to 1×10⁷ cells per mouse in a volume of 100 μL using a 27 gauge needle. Mice are then ear tagged and tumors are measured twice weekly. Candidate modulator treatment is initiated on the day the mean tumor weight reaches 100 mg. Candidate modulator is delivered IV, SC, IP, or PO by bolus administration. Depending upon the pharmacokinetics of each unique candidate modulator, dosing can be performed multiple times per day. The tumor weight is assessed by measuring perpendicular diameters with a caliper and calculated by multiplying the measurements of diameters in two dimensions. At the end of the experiment, the excised tumors maybe utilized for biomarker identification or further analyses. For immunohistochemistry staining, xenograft tumors are fixed in 4% paraformaldehyde, 0.1M phosphate, pH 7.2, for 6 hours at 4° C., immersed in 30% sucrose in PBS, and rapidly frozen in isopentane cooled with liquid nitrogen.

In another preferred embodiment, tumorogenicity is monitored using a hollow fiber assay, which is described in U.S. Pat. No. 5,698,413. Briefly, the method comprises implanting into a laboratory animal a biocompatible, semi-permeable encapsulation device containing target cells, treating the laboratory animal with a candidate modulating agent, and evaluating the target cells for reaction to the candidate modulator. Implanted cells are generally human cells from a pre-existing tumor or a tumor cell line known to be angiogenic. After an appropriate period of time, generally around six days, the implanted samples are harvested for evaluation of the candidate modulator. Tumorogenicity and modulator efficacy may be evaluated by assaying the quantity of viable cells present in the macrocapsule, which can be determined by tests known in the art, for example, MTT dye conversion assay, neutral red dye uptake, trypan blue staining, viable cell counts, the number of colonies formed in soft agar, the capacity of the cells to recover and replicate in vitro, etc. Other assays specific to angiogenesis, as are known in the art and described herein, may also be used.

In another preferred embodiment, a tumorogenicity assay use a transgenic animal, usually a mouse, carrying a dominant oncogene or tumor suppressor gene knockout under the control of tissue specific regulatory sequences; these assays are generally referred to as transgenic tumor assays. In a preferred application, tumor development in the transgenic model is well characterized or is controlled. In an exemplary model, the “RIP1-Tag2” transgene, comprising the SV40 large T-antigen oncogene under control of the insulin gene regulatory regions is expressed in pancreatic beta cells and results in islet cell carcinomas (Hanahan D, 1985, Nature 315:115-122; Parangi S et al, 1996, Proc Natl Acad Sci USA 93: 2002-2007; Bergers G et al, 1999, Science 284:808-812). An “angiogenic switch,” occurs at approximately five weeks, as normally quiescent capillaries in a subset of hyperproliferative islets become angiogenic. The RIP1-TAG2 mice die by age 14 weeks. Candidate modulators may be administered at a variety of stages, including just prior to the angiogenic switch (e.g., for a model of tumor prevention), during the growth of small tumors (e.g., for a model of intervention), or during the growth of large and/or invasive tumors (e.g., for a model of regression). Tumorogenicity and modulator efficacy can be evaluating life-span extension and/or tumor characteristics, including number of tumors, tumor size, tumor morphology, vessel density, apoptotic index, etc.

Diagnostic and Therapeutic Uses

Specific MAP2K6-modulating agents are useful in a variety of diagnostic and therapeutic applications where disease or disease prognosis is related to defects in branching morphogenesis, such as angiogenic, apoptotic, or cell proliferation disorders. Accordingly, the invention also provides methods for modulating branching morphogenesis in a cell, preferably a cell pre-determined to have defective or impaired branching morphogenesis function (e.g. due to overexpression, underexpression, or misexpression of branching morphogenesis, or due to gene mutations), comprising the step of administering an agent to the cell that specifically modulates MAP2K6 activity. Preferably, the modulating agent produces a detectable phenotypic change in the cell indicating that the branching morphogenesis function is restored. The phrase “function is restored”, and equivalents, as used herein, means that the desired phenotype is achieved, or is brought closer to normal compared to untreated cells. For example, with restored branching morphogenesis function, cell proliferation and/or progression through cell cycle may normalize, or be brought closer to normal relative to untreated cells. The invention also provides methods for treating disorders or disease associated with impaired branching morphogenesis function by administering a therapeutically effective amount of a MAP2K6-modulating agent that modulates branching morphogenesis. The invention further provides methods for modulating MAP2K6 function in a cell, preferably a cell pre-determined to have defective or impaired MAP2K6 function, by administering a MAP2K6-modulating agent. Additionally, the invention provides a method for treating disorders or disease associated with impaired MAP2K6 function by administering a therapeutically effective amount of a MAP2K6-modulating agent. In certain embodiments the impaired MAP2K6 function is attributable to impaired particular ones.

The discovery that MAP2K6 is implicated in branching morphogenesis provides for a variety of methods that can be employed for the diagnostic and prognostic evaluation of diseases and disorders involving defects in branching morphogenesis and for the identification of subjects having a predisposition to such diseases and disorders.

Various expression analysis methods can be used to diagnose whether MAP2K6 expression occurs in a particular sample, including Northern blotting, slot blotting, ribonuclease protection, quantitative RT-PCR, and microarray analysis. (e.g., Current Protocols in Molecular Biology (1994) Ausubel F M et al., eds., John Wiley & Sons, Inc., chapter 4; Freeman W M et al., Biotechniques (1999) 26:112-125; Kallioniemi O P, Ann Med 2001, 33:142-147; Blohm and Guiseppi-Elie, Curr Opin Biotechnol 2001, 12:41-47). Tissues having a disease or disorder implicating defective branching morphogenesis signaling that express a MAP2K6, are identified as amenable to treatment with a MAP2K6 modulating agent. In a preferred application, the branching morphogenesis defective tissue overexpresses a MAP2K6 relative to normal tissue. For example, a Northern blot analysis of mRNA from tumor and normal cell lines, or from tumor and matching normal tissue samples from the same patient, using full or partial MAP2K6 cDNA sequences as probes, can determine whether particular tumors express or overexpress MAP2K6. Alternatively, the TaqMan® is used for quantitative RT-PCR analysis of MAP2K6 expression in cell lines, normal tissues and tumor samples (PE Applied Biosystems).

Various other diagnostic methods may be performed, for example, utilizing reagents such as the MAP2K6 oligonucleotides, and antibodies directed against a MAP2K6, as described above for: (1) the detection of the presence of MAP2K6 gene mutations, or the detection of either over- or under-expression of MAP2K6 mRNA relative to the non-disorder state; (2) the detection of either an over- or an under-abundance of MAP2K6 gene product relative to the non-disorder state; and (3) the detection of perturbations or abnormalities in the signal transduction pathway mediated by MAP2K6.

Thus, in a specific embodiment, the invention is drawn to a method for diagnosing a disease or disorder in a patient that is associated with alterations in MAP2K6 expression, the method comprising: a) obtaining a biological sample from the patient; b) contacting the sample with a probe for MAP2K6 expression; c) comparing results from step (b) with a control; and d) determining whether step (c) indicates a likelihood of the disease or disorder. Preferably, the disease is cancer, most preferably a cancer as shown in TABLE 1. The probe may be either DNA or protein, including an antibody.

### EXAMPLES

The following experimental section and examples are offered by way of illustration and not by way of limitation.

I. Analysis of Vasculature Defects Associated with Dr_map2k3 Loss of Function

Wild type, one-cell stage embryos from the Tübingen strain were treated with antisense morpholino oligonucleotide (PMOs) that targeted the 5′UTR and/or start codon of predicted zebrafish genes. PMOs were dissolved at a concentration of 3 mg/mL in injection buffer (0.4 mM MgSO₄, 0.6 mM CaCl₂, 0.7 mM KCl, 58 mM NaCl, 25 mM Hepes [pH 7.6]); a total of 1.5 nL (=4.5 ng) was injected into zebrafish embryos at the 1-cell stage.

Larvae were fixed at 4 days post fertilization (dpf) in 4% para-formaldehyde in phosphate-buffered saline (PBS) for 30 minutes. Fixed larvae were dehydrated in methanol and stored over night at −20° C. After permeabilization in acetone (30 minutes at −20° C.), embryos were washed in PBS and incubated in the staining buffer (100 mM Tris-HCl [pH 9.5], 50 mM MgCl₂, 100 mM NaCl, 0.1% Tween-20) for 45 minutes. Staining reaction was started by adding 2.25 μl nitro blue tetrazolium (NBT, Sigma) and 1.75 μl 5-bromo-4-chloro-3-indolyl phosphate (BCIP, Sigma) per ml of staining buffer (stock solutions: 75 mg/ml NBT in 70% N,N-dimethylformamide, 50 mg/ml BCIP in N,N-dimethylformamide).

The fixed specimens were scanned for changes in blood vessel formation, in particular, for any pro-angiobenic, anti-angiogenic, vasculogenic or vessel patterning phenotypes, among others. Other phenotypic changes resulting from the PMO treatment were also noted. Hits were “Confirmed” when the phenotype was seen for 2^{nd} time in an independent injection of the PMO. Hits were “Characterized” when phenotype was seen for a 3^{rd} time by angiography, to visualize the vascular anatomy. Treatment of embryos with a PMO which targets the Dr_map2k3 messenger RNA produced defects in larval vasculature. Orthologs of the Dr_map2k3 are referred to herein as MAP2K6.

BLAST analysis (Altschul et al., supra) was employed to identify orthologs of zebrafish modifiers. For example, representative sequence from MAP2K6, GI# 14589899 (SEQ ID NO:1) shares 87% amino acid identity with the zebrafish Dr_map2k3.

Various domains, signals, and functional subunits in proteins were analyzed using the PSORT (Nakai K., and Horton P., Trends Biochem Sci, 1999, 24:34-6; Kenta Nakai, Protein sorting signals and prediction of subcellular localization, Adv. Protein Chem. 54, 277-344 (2000)), PFAM (Bateman A., et al., Nucleic Acids Res, 1999, 27:260-2), SMART (Ponting C P, et al., SMART: identification and annotation of domains from signaling and extracellular protein sequences. Nucleic Acids Res. 1999 Jan. 1; 27(1):229-32), TM-HMM (Erik L. L. Sonnhammer, Gunnar von Heijne, and Anders Krogh: A hidden Markov model for predicting transmembrane helices in protein sequences. In Proc. of Sixth Int. Conf. on Intelligent Systems for Molecular Biology, p 175-182 Ed J. Glasgow, T. Littlejohn, F. Major, R. Lathrop, D. Sankoff, and C. Sensen Menlo Park, Calif.: AAAI Press, 1998), and clust (Remm M, and Sonnhammer E. Classification of transmembrane protein families in the Caenorhabditis elegans genome and identification of human orthologs. Genome Res. 2000 November; 10(11):1679-89) programs. For example, the kinase domain (PFAM 00069) of MAP2K6 from GI# 14589900 (SEQ D NO:3) is located at approximately amino acid residues 53 to 314.

II. Zebrafish “Negative” & “Positive” Secondary Assays for Morpholino (PMO) Screen Hits

Zebrafish “Negative” secondary assays are used to determine whether the effects seen on the vasculature with the morpholino knockdown is a primary effect on the vasculature vs. a secondary effect caused by a general patterning defect. Zebrafish “Positive” secondary assays provide pathway and/or mechanistic information about the gene target as well as cell and tissue specificity of its activity.

Negative assay #1—Patterning vs. vascular defects. Whole mount stains are done with muscle-specific antibody mAb MF20 (acto-myosin) to evaluate whether there is a general patterning defect caused by the gene knockdown.

Negative assay #2—Neuronal vs. vascular defects. Whole mount stains with a neuronal-specific antibody (anti-acetylated tubulin) to evaluate whether there is a underlying neuronal patterning defect that may cause a secondary vascular phenotype.

Negative assay #3—Tissue dystrophic or necrotic vs. vascular defects. Live observation of morphology under Nomarski optics (at day 1-4 of development following PMO injection) to evaluate the extent of tissue apoptosis/necrosis induced by gene knockdown.

Negative assay #4—Vascular or Hematopoietic Marker Expression (in situ hybridization). In situ hybridization w/fli1 gene, which stains developing vessels, is done at day 2 of development to evaluate whether the phenotype observed at day 4 results from a vascular development defect vs. vascular maintenance defect.

Positive assay #5: Anti-Angiogenesis pathway interactions with VEGF-Receptor (KDR) and with Target gene PMOs. Target gene PMO with PMO to knockdown the KDR (VEGFR2) gene are co-injected to evaluate whether the target functions in the VEGF pathway.

III. High-Throughput In Vitro Fluorescence Polarization Assay

Fluorescently-labeled MAP2K6 peptide/substrate are added to each well of a 96-will microtiter plate, along with a test agent in a test buffer (10 mM HEPES, 10 mM MgCl, 6 mM magnesium chloride, pH 7.6). Changes in fluorescence polarization, determined by using a Fluorolite FPM-2 Fluorescence Polarization Microtiter System (Dynatech Laboratories, Inc), relative to control values indicates the test compound is a candidate modifier of MAP2K6 activity.

IV. High-Throughput In Vitro Binding Assay.

³³P-labeled MAP2K6 peptide is added in an assay buffer (100 mM KCl, 20 mM HEPES pH 7.6, 1 mM MgCl₂, 1% glycerol, 0.5% NP-40, 50 mM beta-mercaptoethanol, 1 mg/ml BSA, cocktail of protease inhibitors) along with a test agent to the wells of a Neutralite-avidin coated assay plate and incubated at 25° C. for 1 hour. Biotinylated substrate is then added to each well and incubated for 1 hour. Reactions are stopped by washing with PBS, and counted in a scintillation counter. Test agents that cause a difference in activity relative to control without test agent are identified as candidate branching morphogenesis modulating agents.

V. Immunoprecipitations and Immunoblotting

For coprecipitation of transfected proteins, 3×10⁶ appropriate recombinant cells containing the MAP2K6 proteins are plated on 10-cm dishes and transfected on the following day with expression constructs. The total amount of DNA is kept constant in each transfection by adding empty vector. After 24 h, cells are collected, washed once with phosphate-buffered saline and lysed for 20 min on ice in 1 ml of lysis buffer containing 50 mM Hepes, pH 7.9, 250 mM NaCl, 20 mM-glycerophosphate, 1 mM sodium orthovanadate, 5 mM p-nitrophenyl phosphate, 2 mM dithiothreitol, protease inhibitors (complete, Roche Molecular Biochemicals), and 1% Nonidet P-40. Cellular debris is removed by centrifugation twice at 15,000×g for 15 min. The cell lysate is incubated with 25 μl of M2 beads (Sigma) for 2 h at 4° C. with gentle rocking.

After extensive washing with lysis buffer, proteins bound to the beads are solubilized by boiling in SDS sample buffer, fractionated by SDS-polyacrylamide gel electrophoresis, transferred to polyvinylidene difluoride membrane and blotted with the indicated antibodies. The reactive bands are visualized with horseradish peroxidase coupled to the appropriate secondary antibodies and the enhanced chemiluminescence (ECL) Western blotting detection system (Amersham Pharmacia Biotech).

VI. Kinase Assay

A purified or partially purified MAP2K6 is diluted in a suitable reaction buffer, e.g., 50 mM Hepes, pH 7.5, containing magnesium chloride or manganese chloride (1-20 mM) and a peptide or polypeptide substrate, such as myelin basic protein or casein (1-10 μg/ml). The final concentration of the kinase is 1-20 nM. The enzyme reaction is conducted in microtiter plates to facilitate optimization of reaction conditions by increasing assay throughput. A 96-well microtiter plate is employed using a final volume 30-100 μl. The reaction is initiated by the addition of ³³P-gamma-ATP (0.5 μCi/ml) and incubated for 0.5 to 3 hours at room temperature. Negative controls are provided by the addition of EDTA, which chelates the divalent cation (Mg2⁺ or Mn²⁺) required for enzymatic activity. Following the incubation, the enzyme reaction is quenched using EDTA. Samples of the reaction are transferred to a 96-well glass fiber filter plate (MultiScreen, Millipore). The filters are subsequently washed with phosphate-buffered saline, dilute phosphoric acid (0.5%) or other suitable medium to remove excess radiolabeled ATP. Scintillation cocktail is added to the filter plate and the incorporated radioactivity is quantitated by scintillation counting (Wallac/Perkin Elmer). Activity is defined by the amount of radioactivity detected following subtraction of the negative control reaction value (EDTA quench).

VII. Expression Analysis

All cell lines used in the following experiments are NCI (National Cancer Institute) lines, and are available from ATCC (American Type Culture Collection, Manassas, Va. 20110-2209). Normal and tumor tissues were obtained from Impath, UC Davis, Clontech, Stratagene, Ardais, Genome Collaborative, and Ambion.

TaqMan analysis was used to assess expression levels of the disclosed genes in various samples.

RNA was extracted from each tissue sample using Qiagen (Valencia, Calif.) RNeasy kits, following manufacturer's protocols, to a final concentration of 50 ng/μl. Single stranded cDNA was then synthesized by reverse transcribing the RNA samples using random hexamers and 500 ng of total RNA per reaction, following protocol 4304965 of Applied Biosystems (Foster City, Calif.).

Primers for expression analysis using TaqMan assay (Applied Biosystems, Foster City, Calif.) were prepared according to the TaqMan protocols, and the following criteria: a) primer pairs were designed to span introns to eliminate genomic contamination, and b) each primer pair produced only one product. Expression analysis was performed using a 7900HT instrument.

Taqman reactions were carried out following manufacturer's protocols, in 25 μl total volume for 96-well plates and 10 μl total volume for 384-well plates, using 300 nM primer and 250 nM probe, and approximately 25 ng of cDNA. The standard curve for result analysis was prepared using a universal pool of human cDNA samples, which is a mixture of cDNAs from a wide variety of tissues so that the chance that a target will be present in appreciable amounts is good. The raw data were normalized using 18S rRNA (universally expressed in all tissues and cells).

For each expression analysis, tumor tissue samples were compared with matched normal tissues from the same patient. A gene was considered overexpressed in a tumor when the level of expression of the gene was 2 fold or higher in the tumor compared with its matched normal sample. In cases where normal tissue was not available, a universal pool of cDNA samples was used instead. In these cases, a gene was considered overexpressed in a tumor sample when the difference of expression levels between a tumor sample and the average of all normal samples from the same tissue type was greater than 2 times the standard deviation of all normal samples (i.e., Tumor−average (all normal samples)>2×STDEV (all normal samples)).

Results are shown in Table 1. Number of pairs of tumor samples and matched normal tissue from the same patient are shown for each tumor type. Percentage of the samples with at least two-fold overexpression for each tumor type is provided. A modulator identified by an assay described herein can be further validated for therapeutic effect by administration to a tumor in which the gene is overexpressed. A decrease in tumor growth confirms therapeutic utility of the modulator. Prior to treating a patient with the modulator, the likelihood that the patient will respond to treatment can be diagnosed by obtaining a tumor sample from the patient, and assaying for expression of the gene targeted by the modulator. The expression data for the gene(s) can also be used as a diagnostic marker for disease progression. The assay can be performed by expression analysis as described above, by antibody directed to the gene target, or by any other available detection method.

TABLE 1

| | |
|---|---|
| SEQ ID NO: | 1 |
| Breast | 6% |
| # of Pairs | 36 |
| Colon | 20% |
| # of Pairs | 41 |
| Head And | 0% |
| Neck | |
| # of Pairs | 13 |
| Kidney | 0% |
| # of Pairs | 21 |
| Liver | 40% |
| # of Pairs | 5 |
| Lung | 18% |
| # of Pairs | 39 |
| Lymphoma | 0% |
| # of Pairs | 2 |
| Ovary | 26% |
| # of Pairs | 19 |
| Pancreas | 22% |
| # of Pairs | 9 |
| Prostate | 29% |
| # of Pairs | 21 |
| Skin | 29% |
| # of Pairs | 7 |
| Stomach | 55% |
| # of Pairs | 11 |
| Testis | 62% |
| # of Pairs | 8 |
| Thyroid | 7% |
| Gland | |
| # of Pairs | 14 |
| Uterus | 40% |
| # of Pairs | 20 |

VIII. Proliferation Assay

Human umbilical endothelial cells (HMVEC) are maintained at 37° C. in flasks or plates coated with 1.5% porcine skin gelatin (300 bloom, Sigma) in Growth medium (Clonetics Corp.) supplemented with 10-20% fetal bovine serum (FBS, Hyclone). Cells are grown to confluency and used up to the seventh passage. Stimulation medium consists of 50% Sigma 99 media and 50% RPMI 1640 with L-glutamine and additional supplementation with 10 μg/ml insulin-transferrin-selenium (Gibco BRL) and 10% FBS. Cell growth is stimulated by incubation in Stimulation medium supplemented with 20 ng/ml of VEGF. Cell culture assays are carried out in triplicate. Cells are transfected with a mixture of 10 μg of pSV7d expression vectors carrying the MAP2K6 or the MAP2K6 coding sequences and 1 μg of pSV2 expression vector carrying the neo resistance gene with the Lipofectin reagent (Life Technologies, Inc.). Stable integrants are selected using 500 μg/ml G418; cloning was carried out by colony isolation using a Pasteur pipette. Transformants are screened by their ability to specifically bind iodinated VEGF. Proliferation assays are performed on growth-arrested cells seeded in 24-well cluster plates. The cell monolayers are incubated in serum-free medium with the modulators and 1 μCi of [3H]thymidine (47 Ci/mmol) for 4 h. The insoluble material is precipitated for 10 min with 10% trichloroacetic acid, neutralized, and dissolved in 0.2 M NaOH, and the radioactivity is counted in a scintillation counter.

What is claimed is:

1. A method of identifying a candidate branching morphogenesis modulating agent, said method comprising the steps of:
(a) providing an assay system comprising a MAP2K6 polypeptide or nucleic acid;
(b) contacting the assay system with a test agent under conditions whereby, but for the presence of the test agent, the system provides a reference activity; and
(c) detecting a test agent-biased activity of the assay system, wherein a difference between the test agent-biased activity and the reference activity identifies the test agent as a candidate branching morphogenesis modulating agent.

2. The method of claim 1 wherein the assay system includes a screening assay comprising a MAP2K6 polypeptide, and the candidate test agent is a small molecule modulator.

3. The method of claim 2 wherein the screening assay is a kinase assay.

4. The method of claim 1 wherein the assay system includes a binding assay comprising a MAP2K6 polypeptide and the candidate test agent is an antibody.

5. The method of claim 1 wherein the assay system includes an expression assay comprising a MAP2K6 nucleic acid and the candidate test agent is a nucleic acid modulator.

6. The method of claim 5 wherein the nucleic acid modulator is an antisense oligomer.

7. The method of claim 6 wherein the nucleic acid modulator is a PMO.

8. The method of claim 1 wherein the assay system comprises cultured cells or a non-human animal expressing MAP2K6,
and wherein the assay system includes an assay that detects an agent-biased change in branching morphogenesis

9. The method of claim 8 wherein the branching morphogenesis is angiogenesis.

10. The method of claim 8 wherein the assay system comprises cultured cells.

11. The method of claim 10 wherein the assay detects an event selected from the group consisting of cell proliferation, cell cycling, apoptosis, tubulogenesis, cell migration, cell sprouting and response to hypoxic conditions.

12. The method of claim 10 wherein the assay detects tubulogenesis or cell migration or cell sprouting, and wherein the assay system comprises the step of testing the cellular response to stimulation with at least two different pro-angiogenic agents.

13. The method of claim 10 wherein the assay detects tubulogenesis or cell migration, and wherein cells are stimulated with an inflammatory angiogenic agent.

14. The method of claim 8 wherein the assay system comprises a non-human animal.

15. The method of claim 14 wherein the assay system includes a matrix implant assay, a xenograft assay, a hollow fiber assay, or a transgenic tumor assay.

16. The method of claim 15 wherein the assay system includes a transgenic tumor assay that includes a mouse comprising a RIP1-Tag2 transgene.

17. The method of claim 1, comprising the additional steps of:
(d) providing a second assay system comprising cultured cells or a non-human animal expressing MAP2K6,
(e) contacting the second assay system with the test agent of (b) or an agent derived therefrom under conditions whereby, but for the presence of the test agent or agent derived therefrom, the system provides a reference activity; and
(f) detecting an agent-biased activity of the second assay system,
wherein a difference between the agent-biased activity and the reference activity of the second assay system confirms the test agent or agent derived therefrom as a candidate branching morphogenesis modulating agent,
and wherein the second assay system includes a second assay that detects an agent-biased change in an activity associated with branching morphogenesis.

18. The method of claim 17 wherein second assay detects an agent-biased change in an activity associated with angiogenesis.

19. The method of claim 17 wherein the second assay system comprises cultured cells.

20. The method of claim 19 wherein the second assay detects an event selected from the group consisting of cell proliferation, cell cycling, apoptosis, tubulogenesis, cell migration, cell sprouting and response to hypoxic conditions.

21. The method of claim 20 wherein the second assay detects tubulogenesis or cell migration or cell sprouting, and wherein the second assay system comprises the step of testing the cellular response to stimulation with at least two different pro-angiogenic agents.

22. The method of claim 20 wherein the assay detects tubulogenesis or cell migration, and wherein cells are stimulated with an inflammatory angiogenic agent.

23. The method of claim 17 wherein the assay system comprises a non-human animal.

24. The method of claim 23 wherein the assay system includes a matrix implant assay, a xenograft assay, a hollow fiber assay, or a transgenic tumor assay.

25. The method of claim 24 wherein the assay system includes a transgenic tumor assay that includes a mouse comprising a RIP1-Tag2 transgene.

26. A method of modulating branching morphogenesis in a mammalian cell comprising contacting the cell with an agent that specifically binds a MAP2K6 polypeptide or nucleic acid.

27. The method of claim 26 wherein the agent is administered to a mammalian animal predetermined to have a pathology associated with branching morphogenesis.

28. The method of claim 26 wherein the agent is a small molecule modulator, a nucleic acid modulator, or an antibody.

29. The method of claim 26 wherein the branching morphogenesis is angiogenesis

30. The method of claim 29 wherein tumor cell proliferation is inhibited.

31. A method for diagnosing a disease in a patient comprising:
(a) obtaining a biological sample from the patient;
(b) contacting the sample with a probe for MBM expression;
(c) comparing results from step (b) with a control; and
(d) determining whether step (c) indicates a likelihood of disease.

32. The method of claim 31 wherein said disease is cancer.

33. The method according to claim 32, wherein said cancer is a cancer as shown in Table 1 as having >25% expression level.


SEQUENCE LISTING (3 sequences)

SEQ ID NO: 1 - DNA, 2924 bp, Homo sapiens
ggcttctggttcggcccacctctgaaggttccagaatcgatagtgaattcgtggttccaagtttggagcttttagctgccagccctggcccatcatgtagctgcagcacagccttccctaacgttgcaactgggggaaaaatcactttccagtctgttttgcaaggtgtgcatttccatcttgattccctgaaagtccatctgctgcatcggtcaagagaaactccacttgcatgaagattgcacgcctgcagcttgcatctttgttgcaaaactagctacagaagagaagcaaggcaaagtcttttgtgctcccctcccccatcaaaggaaaggggaaaatgtctcagtcgaaaggcaagaagcgaaaccctggccttaaaattccaaaagaagcatttgaacaacctcagaccagttccacaccacctcgagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatgtgtggatctgcatggagctcatggatacatcactagataaattctacaaacaagttattgataaaggccagacaattccagaggacatcttagggaaaatagcagtttctattgtaaaagcattagaacatttacatagtaagctgtctgtcattcacagagacgtcaagccttctaatgtactcatcaatgctctcggtcaagtgaagatgtgcgattttggaatcagtggctacttggtggactctgttgctaaaacaattgatgcaggttgcaaaccatacatggcccctgaaagaataaacccagagctcaaccagaagggatacagtgtgaagtctgacatttggagtctgggcatcacgatgattgagttggccatccttcgatttccctatgattcatggggaactccatttcagcagctcaaacaggtggtagaggagccatcgccacaactcccagcagacaagttctctgcagagtttgttgactttacctcacagtgcttaaagaagaattccaaagaacggcctacatacccagagctaatgcaacatccatttttcaccctacatgaatccaaaggaacagatgtggcatcttttgtaaaactgattcttggagactaaaaagcagtggacttaatcggttgaccctactgtggattggtgggtttcggggtgaagcaagttcactacagcatcaatagaaagtcatctttgagataatttaaccctgcctctcagagggttttctctcccaattttctttttactccccctcttaagggggccttggaatctatagtatagaatgaactgtctagatggatgaattatgataaaggcttaggacttcaaaaggtgattaaatatttaatgatgtgtcatatgagtcctcaagcttctcagacttctcttattctttacaaaatgaatgcattggccctgacaaaaaggtgctacggtagtgatgaaattataagtagatttgtagtttgtcccatttattattttaatatttatgtttaagtgcttggttgaaaagattccattttatacaagaagggagattcaaaaaaaaaatataaggttgggttagcaatatttatagggcttttattttttaagttcaattgtgtctgtggtccagaagaaattatttaatatgcatctttgagaatattataaaaatatcaaaaaggagctcttcttgtgaaatgtctgttccagctgttgtgactgctgccatttttggaaacatctgcccaatcctgggtgatcaccacatcttttaggggaagtgacaagatgctctggtcatactctttttcccaactttggaaaacataaaaatcactcatataacagctcaaagagtaaaacatttggttcttctgacacttgtggtatagtattagtggaaagtgatttgtaatatgattttatatccacctacctattcatctacctgtgtgtatgtgtgtgtttgtgtgtctatttggcaattcacaagtcctgccaagtggtttctatgagcatctctgtttggtaaggaggacaattgtcagttttgagggggacatgtgttaaatcacagaaaaaaatggtgccttcttctgcgtttgtccctcctgccatgtgtaagttgtaaggattgcctttgtagttaatgtactctttggctttgtttgtttgttttcttcttcagtgaagcagccttactattcatagaagggctagaataggagaaaatgaaaggtagtgagtaattctttgataagatgaggaaataatgggaaaggttgaattaattcctgggcatggactaccagatgaccacaagttgcgttgaggccgcatctttcttcagcagcgtgcaatagctggctcctctataggagatgagcttcattgggagttcctagcaagttgactaaacagcaaaagttctttctcgtgggtaaatatacccacaggttctatgatttgtagctctaggtttcttgatgatcaaggagtgaagtaattgacagggaaaatatagacctatgataaataaccaggaagcattgcttttggacaaggaagaacagagggttttgattttaaaaagaagaaaaaaaaaccttattttttctttcttggcctcaagttcaatatggagaggattgcttccctgaatcctctcttccttccccttttagag

SEQ ID NO: 2 - DNA, 2820 bp, Homo sapiens
gcagagtgttgctgtgtgtgcttgtgatttgtattttatttgatgtaaacgtgaaggcagagtattttctaacactgtaattcaactaggttttgtgtctcctggatctatttttttttcttgttgttctgaggagctgatatacttggaaatattaggtttaagatatgcagatgtccaacttatatacatagtcaagggtttagagtctggagacaggaggctggcaatttcaactagggggcaggtcaggcaagaagcgaaaccctggccttaaaattccaaaagaagcatttgaacaacctcagaccagttccacaccacctcgagatttagactccaaggcttgcatttctattggaaatcagaactttgaggtgaaggcagatgacctggagcctataatggaactgggacgaggtgcgtacggggtggtggagaagatgcggcacgtgcccagcgggcagatcatggcagtgaagcggatccgagccacagtaaatagccaggaacagaaacggctactgatggatttggatatttccatgaggacggtggactgtccattcactgtcaccttttatggcgcactgtttcgggagggtgatatgtggatctgcatggagctcatggatacatcactagataaattctacaaacaagttattgataaaggccagacaattccagaggacatcttagggaaaatagcagtttctattgtaaaagcattagaacatttacatagtaagctgtctgtcattcacagagacgtcaagccttctaatgtactcatcaatgctctcggtcaagtgaagatgtgcgattttggaatcagtggctacttggtggactctgttgctaaaacaattgatgcaggttgcaaaccatacatggcccctgaaagaataaacccagagctcaaccagaagggatacagtgtgaagtctgacatttggagtctgggcatcacgatgattgagttggccatccttcgatttccctatgattcatggggaactccatttcagcagctcaaacaggtggtagaggagccatcgccacaactcccagcagacaagttctctgcagagtttgttgactttacctcacagtgcttaaagaagaattccaaagaacggcctacatacccagagctaatgcaacatccatttttcaccctacatgaatccaaaggaacagatgtggcatcttttgtaaaactgattcttggagactaaaaagcagtggacttaatcggttgaccctactgtggattggtgggtttcggggtgaagcaagttcactacagcatcaatagaaagtcatctttgagataatttaaccctgcctctcagagggttttctctcccaattttctttttactccccctcttaagggggccttggaatctatagtatagaatgaactgtctagatggatgaattatgataaaggcttaggacttcaaaaggtgattaaatatttaatgatgtgtcatatgagtcctcaagcttctcagacttctcttattctttacaaaatgaatgcattggccctgacaaaaaggtgctacggtagtgatgaaattataagtagatttgtagtttgtcccatttattattttaatatttatgtttaagtgcttggttgaaaagattccattttatacaagaagggagattcaaaaaaaaaatataaggttgggttagcaatatttatagggcttttattttttaagttcaattgtgtctgtggtccagaagaaattatttaatatgcatctttgagaatattataaaaatatcaaaaaggagctcttcttgtgaaatgtctgttccagctgttgtgactgctgccatttttggaaacatctgcccaatcctgggtgatcaccacatcttttaggggaagtgacaagatgctctggtcatactctttttcccaactttggaaaacataaaaatcactcatataacagctcaaagagtaaaacatttggttcttctgacacttgtggtatagtattagtggaaagtgatttgtaatatgattttatatccacctacctattcatctacctgtgtgtatgtgtgtgtttgtgtgtctatttggcaattcacaagtcctgccaagtggtttctatgagcatctctgtttggtaaggaggacaattgtcagttttgagggggacatgtgttaaatcacagaaaaaaatggtgccttcttctgcgtttgtccctcctgccatgtgtaagttgtaaggattgcctttgtagttaatgtactctttggctttgtttgtttgttttcttcttcagtgaagcagccttactattcatagaagggctagaataggagaaaatgaaaggtagtgagtaattctttgataagatgaggaaataatgggaaaggttgaattaattcctgggcatggactaccagatgaccacaagttgcgttgaggccgcatctttcttcagcagcgtgcaatagctggctcctctataggagatgagcttcattgggagttcctagcaagttgactaaacagcaaaagttctttctcgtgggtaaatatacccacaggttctatgatttgtagctctaggtttcttgatgatcaaggagtgaagtaattgacagggaaaatatagacctatgataaataaccaggaagcattgcttttggacaaggaagaacagagggttttgattttaaaaagaagaaaaaaaaaccttattttttctttcttggcctcaagttcaatatggagaggattgcttccctgaatcctctcttccttccccttttagag

SEQ ID NO: 3 - protein, 334 aa, Homo sapiens
MSQSKGKKRNPGLKIPKEAFEQPQTSSTPPRDLDSKACISIGNQNFEVKADDLEPIMELGRGAYGVVEKMRHVPSGQIMAVKRIRATVNSQEQKRLLMDLDISMRTVDCPFTVTFYGALFREGDVWICMELMDTSLDKFYKQVIDKGQTIPEDILGKIAVSIVKALEHLHSKLSVIHRDVKPSNVLINALGQVKMCDFGISGYLVDSVAKTIDAGCKPYMAPERINPELNQKGYSVKSDIWSLGITMIELAILRFPYDSWGTPFQQLKQVVEEPSPQLPADKFSAEFVDFTSQCLKKNSKERPTYPELMQHPFFTLHESKGTDVASFVKLILGD